(19) United States
(12) Patent Application Publication
(10) Pub. No.: US 2023/0088367 A1
(43) Pub. Date: Mar. 23, 2023

(54) OXYGEN CONCENTRATOR WITH REMOVABLE SIEVE BED CANISTER ASSEMBLY

(71) Applicant: ResMed Asia Pte. Ltd., Singapore (SG)

(72) Inventors: Kean Wah LOW, Singapore (SG); Kyi Thu MAUNG, Singapore (SG); Wei Liem NGO, Singapore (SG); Tirza SUMITRO, Singapore (SG)

(73) Assignee: ResMed Asia Pte. Ltd., Singapore (SG)

(21) Appl. No.: 17/799,329

(22) PCT Filed: Feb. 25, 2021
(86) PCT No.: PCT/SG2021/050092

§ 371 Date: Aug. 12, 2022

Related U.S. Application Data
(60) Provisional application No. 62/985,688, filed on Mar. 5, 2020.

(51) Int. Cl. A61M 16/00

(52) U.S. Cl. CPC A61M 16/0051; A61M 16/024; A61M 16/049

(57) ABSTRACT

An oxygen concentrator may have a compressor to feed a feed gas for sieve bed(s) via a first manifold, an accumulator to receive enriched air from the bed(s) via a second manifold. It may include an outer housing for the manifolds, the compressor, and the accumulator. The housing may include an access portal to a compartment therein, for removably receiving the bed(s) as a canister assembly. The first manifold may be adjacent to the compartment and have inlet coupling(s) for removably coupling respectively with inlet(s) of the canister assembly. The inlet coupling(s) may each have a first central axis. The second manifold may be adjacent to the compartment and have outlet coupling(s) for removably coupling respectively with outlet(s) of the canister assembly. The outlet coupling(s) may each having a second central axis. The first and second central axes may form any one of an obtuse, acute, or right angle.

### CROSS REFERENCE TO RELATED APPLICATIONS

The present disclosure claims priority from U.S. Provisional Patent Application Ser. No. 62/985,688 filed on Mar. 5, 2020, the entire disclosure of which is hereby incorporated by reference.

### FIELD OF THE TECHNOLOGY

The present technology relates generally to methods and apparatus for treating respiratory disorders, such as those involving gas adsorption or controlled pressure and/or vacuum swing adsorption. Such methods and apparatus may be implemented in an oxygen concentrator using one or more sieve beds, which may be removable such as when configured as a canister assembly.

### BACKGROUND

### The Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Examples of respiratory disorders include respiratory failure, Obesity Hyperventilation Syndrome (OHS), Chronic Obstructive Pulmonary Disease (COPD), Neuromuscular Disease (NMD) and Chest wall disorders.

Respiratory failure is an umbrella term for respiratory disorders in which the lungs are unable to inspire sufficient oxygen or exhale sufficient CO₂ to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

### Therapies

Various respiratory therapies have been used to treat one or more of the above respiratory disorders.

### Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube. In some forms, the comfort and effectiveness of these therapies may be improved.

### Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched air. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient's peak inspiratory flow rate. HFT has been used to treat respiratory failure, COPD, and other respiratory disorders. One mechanism of action is that the high flow rate of air at the airway entrance improves ventilation efficiency by flushing, or washing out, expired CO₂ from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### Respiratory Therapy Systems

These respiratory therapies may be provided by a respiratory therapy system or device. Such systems and devices may also be used to screen, diagnose, or monitor a condition without treating it.

A respiratory therapy system may comprise an oxygen source, an air circuit, and a patient interface.

### Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal LTOT, the patient interface is configured to insufflate the nares but specifically to avoid a complete seal. One example of such a patient interface is a nasal cannula.

An oxygen concentrator may control oxygen enriched air release in a pulsed or demand mode. This may be achieved by delivering the oxygen as a series of pulses, where each pulse or “bolus” may be timed to coincide with inspiration. Such a mode is typically controlled by actuating a pneumatic valve that releases oxygen enriched air for a fixed time. The fixed time is calibrated to be associated with a desired or target bolus volume. However, as such a fixed-time bolus release process does not always achieve the target bolus volume (e.g., due to system characteristics such as compressor variability, as well as aspects of the adsorption process such as the PSA cycle, adsorbent condition, air filter condition etc.), the pneumatic valve may also be operated in a variable manner to regulate the delivered bolus volume more closely to the target volume.

### Air Circuit

An air circuit is a conduit or a tube constructed and arranged to allow, in use, a flow of breathable gas to travel between two components of a respiratory therapy system such as the oxygen source and the patient interface. In some cases, there may be separate limbs of the air circuit for inhalation and exhalation. In other cases, a single limb air circuit is used for both inhalation and exhalation.

### Oxygen Source

Experts in this field have recognized that exercise for respiratory failure patients provides long term benefits that slow the progression of the disease, improve quality of life and extend patient longevity. Most stationary forms of exercise like tread mills and stationary bicycles, however, are too strenuous for these patients. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and are heavy, weighing about 50 pounds when mounted.

Oxygen concentrators have been in use for about 50 years to supply oxygen for respiratory therapy. Traditional oxygen concentrators have been bulky and heavy making ordinary ambulatory activities with them difficult and impractical. Recently, companies that manufacture large stationary oxygen concentrators began developing portable oxygen concentrators (POCs). The advantage of POCs is that they can produce a theoretically endless supply of oxygen. In order to make these devices small for mobility, the various systems necessary for the production of oxygen enriched air are condensed. POCs seek to utilize their produced oxygen as efficiently as possible, in order to minimise weight, size, and power consumption. This may be achieved by delivering the oxygen as series of pulses or “boluses,” each bolus timed to coincide with the onset of inhalation. Such a mode of operation may be implemented with a conserver. The therapy mode is known as pulsed oxygen delivery (POD) or demand mode, in contrast with traditional continuous flow delivery more suited to stationary oxygen concentrators.

Oxygen concentrators may implement processes such as vacuum swing adsorption (VSA), pressure swing adsorption (PSA), or vacuum pressure swing adsorption (VPSA). For example, oxygen concentrators, e.g., POCs, may work based on depressurization (e.g., vacuum operation) and/or pressurization (e.g., compressor operation) in a swing adsorption process (e.g., Vacuum Swing Adsorption VSA, Pressure Swing Adsorption PSA or Vacuum Pressure Swing Adsorption VPSA, each of which are referred to herein as a “swing adsorption process”). For example, an oxygen concentrator may control a process of pressure swing adsorption (PSA). Pressure swing adsorption involves using a compressor to increase gas pressure inside a canister that contains particles of a gas separation adsorbent that attracts nitrogen more strongly than it does oxygen. Such a canister when containing a mass of adsorbent, such as a layer of adsorbent, may serve as a sieve bed. Ambient air usually includes approximately 78% nitrogen and 21% oxygen with the balance comprised of argon, carbon dioxide, water vapor and other trace gases. If a feed gas mixture such as air, for example, is passed under pressure through a sieve bed, part or all of the nitrogen will be adsorbed by the sieve bed, and the gas coming out of the vessel will be enriched in oxygen. When the sieve bed reaches the end of its capacity to adsorb nitrogen, it can be regenerated by reducing the pressure, thereby releasing the adsorbed nitrogen. It is then ready for another “PSA cycle” of producing oxygen enriched air. By alternating pressurization cycles of the canisters in a two-canister system, one canister can be concentrating oxygen (the so-called “adsorption phase”) while the other canister is being purged (the “purge phase”). This alternation results in a near-continuous separation of the oxygen from the nitrogen. In this manner, oxygen can be continuously concentrated out of the air for a variety of uses include providing LTOT to users.

Vacuum swing adsorption (VSA) provides an alternative gas separation technique. VSA typically draws the gas through the separation process of the sieve beds using a vacuum such as a compressor configured to create a vacuum within the sieve beds. Vacuum Pressure Swing Adsorption (VPSA) may be understood to be a hybrid system using a combined vacuum and pressurization technique. For example, a VPSA system may pressurize the sieve beds for the separation process and also apply a vacuum for purging of the beds.

Even if the sieve beds are alternated according to the PSA, VSA, or VPSA processes discussed above, they will ultimately retain some of the adsorbed nitrogen after every cycle. As a result, the overall capacity of a sieve bed to adsorb nitrogen will decrease with use, eventually requiring the sieve beds to be replaced. There is thus a need for designs and mechanisms that enable easy removal and installation of sieve beds, such as in relation to the canisters, from and into the oxygen concentrator for their replacement that may be performed by typical users of the oxygen concentrator such as without requiring tools for the removal and replacement of the canisters/module.

For example, the adsorbent of a sieve bed in a POC may typically be replaced, such as by removal of the canister of the sieve bed, at regular intervals (typically once a year) to ensure consistent and high-purity oxygen generation. In order to minimize or eliminate the cost and effort required for a user to return the POC device for such services and the need for service center to replace the sieve bed, POC use can be improved by a user-removable sieve bed design. A removable sieve bed design enables sieve bed replacement to be done by the user, usually through a plug and play concept. In order to enable a plug and play design, a removable sieve bed should have a standalone construction that enables easy assembly, easy replacement, an effective pneumatic connection for gas exchange, and a secure retention within the POC during operation. In addition, the routing of the pneumatic system within the device should ensure efficient and balanced routing to ensure a reliable operation of the sieve bed.

### SUMMARY OF THE TECHNOLOGY

Examples of the present technology may provide an apparatus for an oxygen concentrator, such as a portable oxygen concentrator. In particular, the technology provides methods and apparatus for a portable oxygen concentrator having a readily replaceable sieve bed canister assembly.

Examples of the present technology that provide a removable sieve bed canister assembly for a portable oxygen concentrator, may provide: (a) an ease of manufacturability compared to other designs; (b) a smaller deadspace when compared to other designs such as those where all inlet and outlet ports are at the same end of a removable canister assembly.

In addition, examples of the present technology that provide a portable oxygen concentrator may provide a mechanism that both establishes pneumatic sealing between the removable sieve bed canister assembly and the portable oxygen concentrator, and secures the removable sieve bed canister assembly within the portable oxygen concentrator.

Some implementations of the present technology may include an oxygen concentrator. The oxygen concentrator may include a first manifold. The oxygen concentrator may include a second manifold. The oxygen concentrator may include a compression system, including a motor operated compressor, configured to feed a feed gas for one or more sieve beds via the first manifold. The oxygen concentrator may include an accumulator configured to receive oxygen enriched air from the one or more sieve beds via the second manifold. The oxygen concentrator may include an outer housing for the first manifold, the second manifold, the compression system, and the accumulator. The outer housing may include an external access portal to a compartment in the outer housing. The compartment may be for removably receiving the one or more sieve beds configured as a removable canister assembly. The first manifold may be in the outer housing adjacent to the compartment. The first manifold may have one or more inlet couplings for removably coupling respectively with one or more inlets of the removable canister assembly. The one or more inlet couplings may each have a first central axis. The second manifold may be in the outer housing adjacent to the compartment. The second manifold may one or more outlet couplings for removably coupling respectively with one or more outlets of the removable canister assembly. The one or more outlet couplings may each have a second central axis. The first central axis and the second central axis may form an angle may include any one of an obtuse angle, an acute angle, or a right angle.

In some implementations, the angle may be in a range of approximately forty degrees to approximately one hundred and thirty-five degrees. The angle may be in a range of approximately fifty degrees to approximately one hundred and twenty-five degrees. The angle may be in a range of approximately sixty degrees to approximately one hundred and fifteen degrees. The angle may be in a range of approximately seventy degrees to approximately one hundred and five degrees. The angle may be approximately ninety degrees.

In some implementations, the oxygen concentrator may further include the removable canister assembly. The removable canister assembly may include at least one canister. Each canister may contain gas separation adsorbent of the one of the one or more sieve beds. Each canister may include an inlet in fluid communication with the contained gas separation adsorbent of the one of the one or more sieve beds. Each canister may include an outlet in fluid communication with the contained gas separation adsorbent of the one of the one or more sieve beds. The inlet and outlet may be arranged at opposite ends of the canister. The inlet may have a third central axis and the outlet may have a fourth central axis. The first central axis may be aligned with the third central axis. The second central axis may be aligned with the fourth central axis.

The removable canister assembly may include a cap portion. The cap portion may include a planar surface for covering one end of the at least one canister. The cap portion may include the inlet of the at least one canister. The third central axis of the inlet of each canister may be generally parallel to the planar surface. The removable canister assembly may include at least two container portions. Each container portion may define a volume for containing gas separation adsorbent of the one or more sieve beds. The container portions may be separable. The container portions may be formed as a unitary structure.

In some implementations, the one or more inlet couplings may include a pneumatically sealable structure to complement a reciprocal structure of the inlet. The one or more outlet couplings may include a pneumatically sealable structure to complement a reciprocal structure of the outlet. The first manifold may be configured to move within the oxygen concentrator to couple and decouple with the inlet for insertion and removal of the removable canister assembly. The first manifold may include one or more valves configured to move with the first manifold. Each of the one or more valves may be configured to gate fluid flow of one of the one or more inlet couplings.

In some implementations, the oxygen concentrator may further include a securing mechanism configured to reversibly engage the first manifold with the removable canister assembly for establishing pneumatic sealing between the first manifold and the removable canister assembly, and/or securing the removable canister assembly within the oxygen concentrator. The securing mechanism may include linkage arms and a lever. The linkage arms and the lever may be configured to move the manifold within a guide frame of the oxygen concentrator. The securing mechanism may further include one or more grip handles for user manipulation of the securing mechanism. The securing mechanism may further include one or more latches for securing the manifold in pneumatic sealing with the removable canister assembly. The securing mechanism may include a knob for securing the manifold in pneumatic sealing with the removable canister assembly.

Some implementations of the present technology may include an oxygen concentrator. The oxygen concentrator may include one or more sieve beds, each containing a gas separation adsorbent. The oxygen concentrator may include a compression system, including a motor operated compressor, configured to feed a feed gas into the one or more sieve beds. The oxygen concentrator may include an accumulator configured to receive oxygen enriched air from the one or more sieve beds. The one or more sieve beds may be configured as a removable canister assembly comprising at least one canister, each canister containing the gas separation adsorbent of one of the one or more sieve beds. Each of the canisters of the removable canister assembly may include an inlet in fluid communication with the contained gas separation adsorbent of the one of the one or more sieve beds. Each of the canisters of the removable canister assembly may include an outlet in fluid communication with the contained gas separation adsorbent of the one of the one or more sieve beds. The oxygen concentrator may include a manifold configured to pneumatically engage at least one inlet or outlet so as to enable fluid flow between the manifold and the at least one inlet or outlet. The oxygen concentrator may include a securing mechanism configured to move the manifold into and out of pneumatic engagement with the at least one inlet or outlet. Moving the manifold into pneumatic engagement with the at least one inlet or outlet may secure each canister within the oxygen concentrator.

The oxygen concentrator may include a guide frame for guiding movement of the manifold. The securing mechanism may include linkage arms and a lever configured to move the manifold within the guide frame of the oxygen concentrator. The securing mechanism may further include one or more grip handles for user manipulation of the manifold. The securing mechanism may further include one or more latches for securing the manifold in pneumatic engagement with the removable canister assembly. The securing mechanism may further include a knob for securing the manifold in pneumatic engagement with the removable canister assembly. The manifold may include a coupling having an orifice configured to receive, within the orifice, a nipple of the at least one inlet or outlet. The manifold may include one or more valves configured to move with the manifold. Each of the one or more valves may be configured to gate a pneumatic path of the manifold.

Some implementations of the present technology may include a removable canister assembly for an oxygen concentrator. The removable canister assembly may include one or more sieve beds containing a gas separation adsorbent. The one or more sieve beds may be configured as one or more canisters, each of the one or more canisters containing gas separation adsorbent. Each of the one or more canisters may include an inlet in fluid communication with the contained gas separation adsorbent. Each of the one or more canisters may include an outlet in fluid communication with the contained gas separation adsorbent. The inlet and outlet may be arranged at opposite ends of the canister. The inlet may have a first central axis and the outlet may have a second central axis. The first central axis and the second central axis may form one of an obtuse angle, an acute angle, or a right angle. Optionally, the removable canister assembly may include an additional canister or more that is/are different from the one or more canisters, such as, a canister that does not include a sieve bed.

In some implementations, the angle may be in a range of approximately forty degrees to approximately one hundred and thirty-five degrees. The angle may be in a range of approximately fifty degrees to approximately one hundred and twenty-five degrees. The angle may be in a range of approximately sixty degrees to approximately one hundred and fifteen degrees. The angle may be in a range of approximately seventy degrees to approximately one hundred and five degrees. The angle may be approximately ninety degrees. The inlet may include a nipple with an inlet port. The outlet may include a nipple with an outlet port. The removable canister assembly may include a cap portion. The cap portion may include a planar surface for covering one end of each of the one or more canisters. The cap portion may further include the outlet of each canister. The second central axis of the outlet may be generally parallel to the planar surface. In some implementations, the cap portion may include the inlet of each canister. The first central axis of the inlet may be generally parallel to the planar surface.

In some implementations, the removable canister assembly may include at least two container portions. Each container portion may define a volume for containing gas separation adsorbent. The container portions may be separable. Alternatively, the container portions may be formed as a unitary structure.

Some implementations of the present technology may include a portable oxygen concentrator apparatus. The portable oxygen concentrator apparatus may include user removable means for containing a gas separation adsorbent. The portable oxygen concentrator apparatus may include means for feeding a feed gas into the user removable means. The portable oxygen concentrator apparatus may include accumulation means for receiving oxygen enriched air from the user removable means. The portable oxygen concentrator apparatus may include securing means. The securing means may be for moving a manifold into and out of pneumatic engagement with at least one inlet or outlet of the user removable means. The securing means may be for securing the user removable means within the portable oxygen concentrator apparatus for use.

Some implementations of the present technology may include a portable oxygen concentrator. The portable oxygen concentrator may include a removable canister assembly that may include a first sieve bed and a second sieve bed, each comprising a canister. The portable oxygen concentrator may include a manifold configured to direct pneumatic flow with each of the first sieve bed and the second sieve bed. The manifold may include a first portion including a first pneumatic path pneumatically coupled with the first sieve bed. The manifold may include a second portion including a second pneumatic path pneumatically coupled with the second sieve bed. The manifold and the removable canister assembly may be substantially symmetrical about a common symmetry plane. The common symmetry plane may be (a) between the first sieve bed and the second sieve bed, and (b) between the first portion and the second portion of the manifold. The manifold may also include a first valve and a second valve. The common symmetry plane may also be between the first valve and the second valve.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### BRIEF DESCRIPTION OF THE DRAWINGS

Advantages of the present technology will become apparent to those skilled in the art with the benefit of the following detailed description of implementations and upon reference to the accompanying drawings in which:

FIG. 1 depicts an oxygen concentrator in accordance with one form of the present technology.

FIG. 2 is a schematic diagram of the components of an oxygen concentrator in accordance with aspects of the technology.

FIG. 3 is a cutaway of an oxygen concentrator in accordance with aspects of the technology.

FIG. 4 is a perspective side view of a compression system of an oxygen concentrator in accordance with aspects of the technology.

FIG. 5 is a side view of a compression system, that includes a heat exchange conduit.

FIG. 6 is a schematic diagram of example outlet components of an oxygen concentrator in accordance with aspects of the technology.

FIG. 7 depicts an outlet conduit for an oxygen concentrator in accordance with aspects of the technology.

FIG. 8 depicts an alternate outlet conduit for an oxygen concentrator in accordance with aspects of the technology.

FIG. 9 is a perspective view of a disassembled canister system for an oxygen concentrator in accordance with aspects of the technology.

FIG. 10 is an end view of the canister system of FIG. 9.

FIG. 11 is an assembled view of the canister system end depicted in FIG. 10.

FIG. 12 is a view of an opposing end of the canister system of FIG. 9 to that depicted in FIGS. 10 and 11.

FIG. 13 is an assembled view of the canister system end depicted in FIG. 12.

FIG. 14 depicts an example control panel for an oxygen concentrator in accordance with aspects of the technology.

FIG. 15A depicts an example removable canister assembly for an oxygen concentrator in accordance with aspects of the technology.

FIG. 15B shows a side view of the removable canister assembly of FIG. 15A.

FIG. 15C depicts container portions of the canisters of the removable canister assembly of FIG. 15A.

FIG. 15D shows a top plan view of the container portions of FIG. 15C.

FIG. 15E shows a front view of the container portions of FIG. 15C.

FIG. 15F shows a bottom, inside view of the container portions of FIG. 15C.

FIG. 15G shows a cap portion of the canisters of the removable canister assembly of FIG. 15A.

FIG. 15H shows an inside plan view of the cap portion of FIG. 15G.

FIG. 15I shows a front view of the cap portion of FIG. 15G.

FIG. 15J shows a side view of the cap portion of FIG. 15G.

FIG. 16A depicts the canister assembly of FIG. 15A installed in a compartment of an oxygen concentrator via a portal to the compartment in accordance with aspects of the technology.

FIG. 16B depicts the compartment of the oxygen concentrator of FIG. 16A without the canister assembly of FIG. 15A.

FIG. 16C depicts the oxygen concentrator of FIG. 16A with an optional removable lid attached to the housing and enclosing the portal to the compartment.

FIGS. 17A and 17B depict an implementation of a securing mechanism for a removable canister assembly in accordance with aspects of the technology.

FIGS. 17C and 17D illustrate an example movable manifold in accordance with some implementations of the present technology such as for implementation or operation with the securing mechanism of FIGS. 17A and 17B.

FIG. 17E shows another view of the movable manifold component of FIGS. 17C and 17D.

FIGS. 18A and 18B depict an implementation of a securing mechanism in accordance with aspects of the technology.

FIGS. 19A and 19B depict an implementation of a securing mechanism in accordance with aspects of the technology.

FIG. 20 illustrates a balancing of the design of a removable canister assembly and a manifold about a common symmetry plane.

### DETAILED DESCRIPTION OF THE IMPLEMENTATIONS

An example adsorption device of the present technology involving an oxygen concentrator may be considered in relation to the examples of the figures. The examples of the present technology may be implemented with any of the following structures and operations.

### Outer Housing

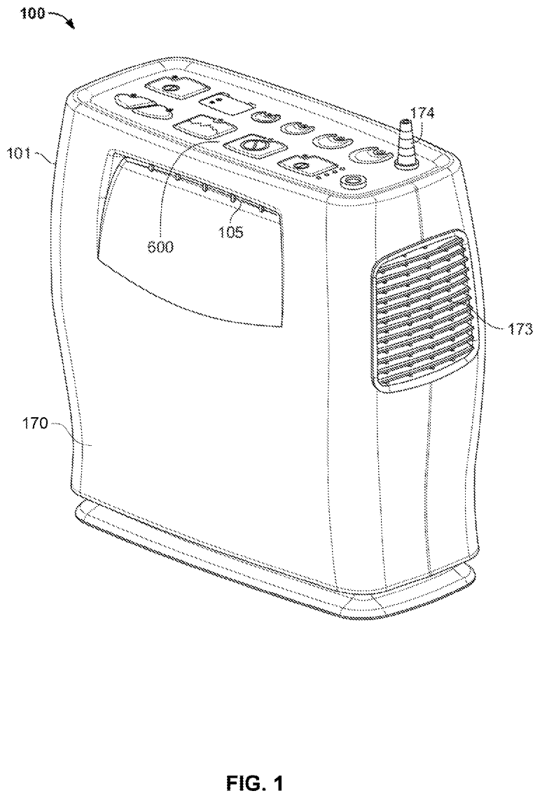

FIG. 1 depicts an implementation of an outer housing 170 of an oxygen concentrator 100. In some implementations, outer housing 170 may be comprised of a light-weight plastic. Outer housing includes compression system inlets 105, cooling system passive inlet 101 and outlet 173 at each end of outer housing 170, outlet port 174, and control panel 600. Inlet 101 and outlet 173 allow cooling air to enter the housing, flow through the housing, and exit the interior of housing 170 to aid in cooling of the oxygen concentrator 100. Compression system inlets 105 allow air to enter the compression system. Outlet port 174 is used to attach a conduit to provide oxygen enriched air produced by the oxygen concentrator 100 to a user.

### Schematic

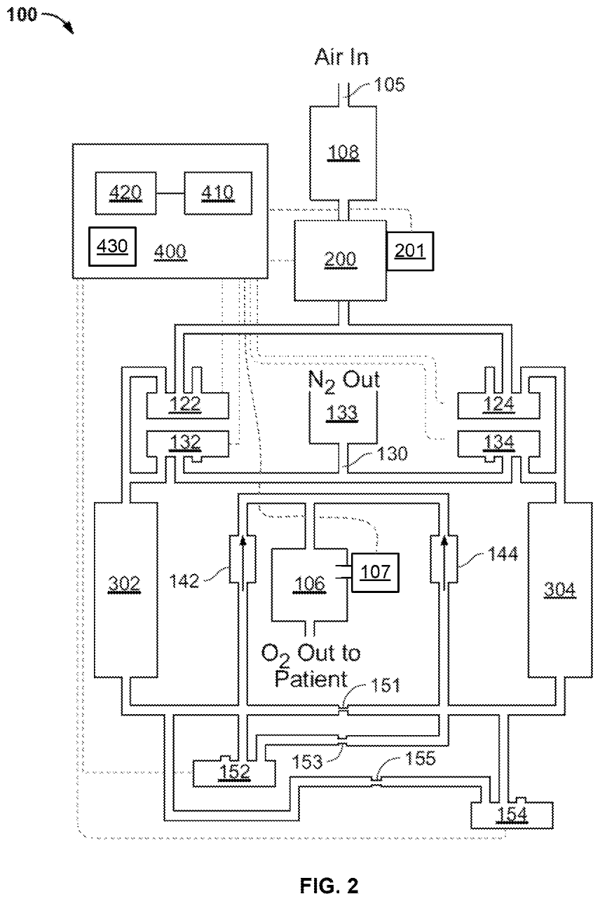

FIG. 2 illustrates a schematic diagram of an oxygen concentrator 100, according to an implementation. Oxygen concentrator 100 may concentrate oxygen within an air stream to provide oxygen enriched air to a user.

Oxygen concentrator 100 may be a portable oxygen concentrator. For example, oxygen concentrator 100 may have a weight and size that allows the oxygen concentrator to be carried by hand and/or in a carrying case. In one implementation, oxygen concentrator 100 has a weight of less than about 20 pounds, less than about 15 pounds, less than about 10 pounds, or less than about 5 pounds. In an implementation, oxygen concentrator 100 has a volume of less than about 1000 cubic inches, less than about 750 cubic inches, less than about 500 cubic inches, less than about 250 cubic inches, or less than about 200 cubic inches.

Oxygen enriched air may be produced from ambient air by pressurising ambient air in canisters 302 and 304, which contain a gas separation adsorbent and are therefore referred to as sieve beds. Gas separation adsorbents useful in an oxygen concentrator are capable of separating at least nitrogen from an air stream to produce oxygen enriched air. Examples of gas separation adsorbents include molecular sieves that are capable of separating nitrogen from an air stream. Examples of adsorbents that may be used in an oxygen concentrator include, but are not limited to, zeolites (natural) or synthetic crystalline aluminosilicates that separate nitrogen from an air stream under elevated pressure. Examples of synthetic crystalline aluminosilicates that may be used include, but are not limited to: OXYSIV adsorbents available from UOP LLC, Des Plaines, Iowa; SYLOBEAD adsorbents available from W. R. Grace & Co, Columbia, Md.; SILIPORITE adsorbents available from CECA S.A. of Paris, France; ZEOCHEM adsorbents available from Zeochem AG, Uetikon, Switzerland; and AgLiLSX adsorbent available from Air Products and Chemicals, Inc., Allentown, Pa.

As shown in FIG. 2, air may enter the oxygen concentrator through air inlet 105. Air may be drawn into air inlet 105 by compression system 200. Compression system 200 may draw in air from the surroundings of the oxygen concentrator and compress the air, forcing the compressed air into one or both canisters 302 and 304. In an implementation, an inlet muffler 108 may be coupled to air inlet 105 to reduce sound produced by air being pulled into the oxygen concentrator by compression system 200. In an implementation, inlet muffler 108 may reduce moisture and sound. For example, a water adsorbent material (such as a polymer water adsorbent material or a zeolite material) may be used to both adsorb water from the incoming air and to reduce the sound of the air passing into the air inlet 105.

Compression system 200 may include one or more compressors configured to compress air. Pressurized air, produced by compression system 200, may be forced into one or both of the canisters 302 and 304. In some implementations, the ambient air may be pressurized in the canisters to a pressure approximately in a range of 13-20 pounds per square inch gauge pressure (psig). Other pressures may also be used, depending on the type of gas separation adsorbent disposed in the canisters.

Coupled to each canister 302/304 are inlet valves 122/124 and outlet valves 132/134. As shown in FIG. 2, inlet valve 122 is coupled to canister 302 and inlet valve 124 is coupled to canister 304. Outlet valve 132 is coupled to canister 302 and outlet valve 134 is coupled to canister 304. Inlet valves 122/124 are used to control the passage of air from compression system 200 to the respective canisters. Outlet valves 132/134 are used to release gas from the respective canisters during a venting process. In some implementations, inlet valves 122/124 and outlet valves 132/134 may be silicon plunger solenoid valves. Other types of valves, however, may be used. Plunger valves offer advantages over other kinds of valves by being quiet and having low slippage.

In some implementations, a two-step valve actuation voltage may be used to control inlet valves 122/124 and outlet valves 132/134. For example, a high voltage (e.g., 24 V) may be applied to an inlet valve to open the inlet valve. The voltage may then be reduced (e.g., to 7 V) to keep the inlet valve open. Using less voltage to keep a valve open may use less power (Power=Voltage*Current). This reduction in voltage minimizes heat buildup and power consumption to extend run time from the power supply 180 (described below). When the power is cut off to the valve, it closes by spring action. In some implementations, the voltage may be applied as a function of time that is not necessarily a stepped response (e.g., a curved downward voltage between an initial 24 V and a final 7 V).

In an implementation, pressurized air is sent into one of canisters 302 or 304 while the other canister is being vented. For example, during use, inlet valve 122 is opened while inlet valve 124 is closed. Pressurized air from compression system 200 is forced into canister 302, while being inhibited from entering canister 304 by inlet valve 124. In an implementation, a controller 400 is electrically coupled to valves 122, 124, 132, and 134. Controller 400 includes one or more processors 410 operable to execute program instructions stored in memory 420. The program instructions configure the controller to perform various predefined methods that are used to operate the oxygen concentrator, such as the methods described in more detail herein. The program instructions may include program instructions for operating inlet valves 122 and 124 out of phase with each other, i.e., when one of inlet valves 122 or 124 is opened, the other valve is closed. During pressurization of canister 302, outlet valve 132 is closed and outlet valve 134 is opened. Similar to the inlet valves, outlet valves 132 and 134 are operated out of phase with each other. In some implementations, the voltages and the durations of the voltages used to open the input and output valves may be controlled by controller 400.

The controller 400 may include a transceiver 430 that may communicate with external devices to transmit data collected by the processor 410 or receive instructions from an external computing device for the processor 410.

Check valves 142 and 144 are coupled to canisters 302 and 304, respectively. Check valves 142 and 144 may be one-way valves that are passively operated by the pressure differentials that occur as the canisters are pressurized and vented, or may be active valves. Check valves 142 and 144 are coupled to the canisters to allow oxygen enriched air produced during pressurization of each canister to flow out of the canister, and to inhibit back flow of oxygen enriched air or any other gases into the canister. In this manner, check valves 142 and 144 act as one-way valves allowing oxygen enriched air to exit the respective canisters during pressurization.

The term “check valve,” as used herein, refers to a valve that allows flow of a fluid (gas or liquid) in one direction and inhibits back flow of the fluid. Examples of check valves that are suitable for use include, but are not limited to: a ball check valve; a diaphragm check valve; a butterfly check valve; a swing check valve; a duckbill valve; an umbrella valve; and a lift check valve. Under pressure, nitrogen molecules in the pressurized ambient air are adsorbed by the gas separation adsorbent in the pressurized canister. As the pressure increases, more nitrogen is adsorbed until the gas in the canister is enriched in oxygen. The nonadsorbed gas molecules (mainly oxygen) flow out of the pressurized canister when the pressure reaches a point sufficient to overcome the resistance of the check valve coupled to the canister. In one implementation, the pressure drop of the check valve in the forward direction is less than 1 psig. The break pressure in the reverse direction is greater than 100 psig. It should be understood, however, that modification of one or more components would alter the operating parameters of these valves. If the forward flow pressure is increased, there is, generally, a reduction in oxygen enriched air production. If the break pressure for reverse flow is reduced or set too low, there is, generally, a reduction in oxygen enriched air pressure.

In an exemplary implementation, canister 302 is pressurized by compressed air produced in compression system 200 and passed into canister 302. During pressurization of canister 302 inlet valve 122 is open, outlet valve 132 is closed, inlet valve 124 is closed and outlet valve 134 is open. Outlet valve 134 is opened when outlet valve 132 is closed to allow substantially simultaneous venting of canister 304 to atmosphere while canister 302 is being pressurized. Canister 302 is pressurized until the pressure in canister is sufficient to open check valve 142. Oxygen enriched air produced in canister 302 exits through check valve and, in one implementation, is collected in accumulator 106.

After some time, the gas separation adsorbent will become saturated with nitrogen and will be unable to separate significant amounts of nitrogen from incoming air. This point is usually reached after a predetermined time of oxygen enriched air production. In the implementation described above, when the gas separation adsorbent in canister 302 reaches this saturation point, the inflow of compressed air is stopped and canister 302 is vented to remove nitrogen. During venting, inlet valve 122 is closed, and outlet valve 132 is opened. While canister 302 is being vented, canister 304 is pressurized to produce oxygen enriched air in the same manner described above. Pressurization of canister 304 is achieved by closing outlet valve 134 and opening inlet valve 124. The oxygen enriched air exits canister 304 through check valve 144.

During venting of canister 302, outlet valve 132 is opened allowing pressurized gas (mainly nitrogen) to exit the canister to atmosphere through concentrator outlet 130. In an implementation, the vented gases may be directed through muffler 133 to reduce the noise produced by releasing the pressurized gas from the canister. As gas is released from canister 302, the pressure in the canister 302 drops, allowing the nitrogen to become desorbed from the gas separation adsorbent. The released nitrogen exits the canister through outlet 130, resetting the canister to a state that allows renewed separation of nitrogen from an air stream. Muffler 133 may include open cell foam (or another material) to muffle the sound of the gas leaving the oxygen concentrator. In some implementations, the combined muffling components/techniques for the input of air and the output of oxygen enriched air may provide for oxygen concentrator operation at a sound level below 50 decibels.

During venting of the canisters, it is advantageous that at least a majority of the nitrogen is removed. In an implementation, at least about 50%, at least about 60%, at least about 70%, at least about 80%, at least about 90%, at least about 95%, at least about 98%, or substantially all of the nitrogen in a canister is removed before the canister is re-used to separate nitrogen from air. In some implementations, a canister may be further purged of nitrogen using an oxygen enriched air stream that is introduced into the canister from the other canister.

In an exemplary implementation, a portion of the oxygen enriched air may be transferred from canister 302 to canister 304 when canister 304 is being vented of nitrogen. Transfer of oxygen enriched air from canister 302 to 304 during venting of canister 304, helps to further purge nitrogen (and other gases) from the canister. In an implementation, oxygen enriched air may travel through flow restrictors 151, 153, and 155 between the two canisters. Flow restrictor 151 may be a trickle flow restrictor. Flow restrictor 151, for example, may be a 0.009D flow restrictor (e.g., the flow restrictor has a radius 0.009″ which is less than the diameter of the tube it is inside). Flow restrictors 153 and 155 may be 0.013D flow restrictors. Other flow restrictor types and sizes are also contemplated and may be used depending on the specific configuration and tubing used to couple the canisters. In some implementations, the flow restrictors may be press fit flow restrictors that restrict air flow by introducing a narrower diameter in their respective tube. In some implementations, the press fit flow restrictors may be made of sapphire, metal or plastic (other materials are also contemplated).

Flow of oxygen enriched air between the canisters is also controlled by use of valve 152 and valve 154. Valves 152 and 154 may be opened for a short duration during the venting process (and may be closed otherwise) to prevent excessive oxygen loss out of the purging canister. Other durations are also contemplated. In an exemplary implementation, canister 302 is being vented and it is desirable to purge canister 302 by passing a portion of the oxygen enriched air being produced in canister 304 into canister 302. A portion of oxygen enriched air, upon pressurization of canister 304, will pass through flow restrictor 151 into canister 302 during venting of canister 302. Additional oxygen enriched air is passed into canister 302, from canister 304, through valve 154 and flow restrictor 155. Valve 152 may remain closed during the transfer process, or may be opened if additional oxygen enriched air is needed. The selection of appropriate flow restrictors 151 and 155, coupled with controlled opening of valve 154 allows a controlled amount of oxygen enriched air to be sent from canister 304 to canister 302. In an implementation, the controlled amount of oxygen enriched air is an amount sufficient to purge canister 302 and minimize the loss of oxygen enriched air through venting valve 132 of canister 302. While this implementation describes venting of canister 302, it should be understood that the same process can be used to vent canister 304 using flow restrictor 151, valve 152 and flow restrictor 153.

The pair of equalization/vent valves 152/154 work with flow restrictors 153 and 155 to optimize the gas flow balance between the two canisters. This may allow for better flow control for venting one of the canisters with oxygen enriched air from the other of the canisters. It may also provide better flow direction between the two canisters. It has been found that, while flow valves 152/154 may be operated as bi-directional valves, the flow rate through such valves varies depending on the direction of fluid flowing through the valve. For example, oxygen enriched air flowing from canister 304 toward canister 302 has a flow rate faster through valve 152 than the flow rate of oxygen enriched air flowing from canister 302 toward canister 304 through valve 152. If a single valve was to be used, eventually either too much or too little oxygen enriched air would be sent between the canisters and the canisters would, over time, begin to produce different amounts of oxygen enriched air. Use of opposing valves and flow restrictors on parallel air pathways may equalize the flow pattern of the oxygen enriched air between the two canisters. Equalising the flow may allow for a steady amount of oxygen enriched air to be available to the user over multiple cycles and also may allow a predictable volume of oxygen enriched air to purge the other of the canisters. In some implementations, the air pathway may not have restrictors but may instead have a valve with a built-in resistance or the air pathway itself may have a narrow radius to provide resistance.

At times, oxygen concentrator may be shut down for a period of time. When an oxygen concentrator is shut down, the temperature inside the canisters may drop as a result of the loss of adiabatic heat from the compression system. As the temperature drops, the volume occupied by the gases inside the canisters will drop. Cooling of the canisters may lead to a negative pressure in the canisters. Valves (e.g., valves 122, 124, 132, and 134) leading to and from the canisters are dynamically sealed rather than hermetically sealed. Thus, outside air may enter the canisters after shutdown to accommodate the pressure differential. When outside air enters the canisters, moisture from the outside air may be adsorbed by the gas separation adsorbent. Adsorption of water inside the canisters may lead to gradual degradation of the gas separation adsorbents, steadily reducing ability of the gas separation adsorbents to produce oxygen enriched air.

In an implementation, outside air may be inhibited from entering canisters after the oxygen concentrator is shut down by pressurising both canisters prior to shutdown. By storing the canisters under a positive pressure, the valves may be forced into a hermetically closed position by the internal pressure of the air in the canisters. In an implementation, the pressure in the canisters, at shutdown, should be at least greater than ambient pressure. As used herein the term “ambient pressure” refers to the pressure of the surroundings in which the oxygen concentrator is located (e.g. the pressure inside a room, outside, in a plane, etc.). In an implementation, the pressure in the canisters, at shutdown, is at least greater than standard atmospheric pressure (i.e., greater than 760 mmHg (Torr), 1 atm, 101,325 Pa). In an implementation, the pressure in the canisters, at shutdown, is at least about 1.1 times greater than ambient pressure; is at least about 1.5 times greater than ambient pressure; or is at least about 2 times greater than ambient pressure.

In an implementation, pressurization of the canisters may be achieved by directing pressurized air into each canister from the compression system and closing all valves to trap the pressurized air in the canisters. In an exemplary implementation, when a shutdown sequence is initiated, inlet valves 122 and 124 are opened and outlet valves 132 and 134 are closed. Because inlet valves 122 and 124 are joined together by a common conduit, both canisters 302 and 304 may become pressurized as air and/or oxygen enriched air from one canister may be transferred to the other canister. This situation may occur when the pathway between the compression system and the two inlet valves allows such transfer. Because the oxygen concentrator operates in an alternating pressurize/venting mode, at least one of the canisters should be in a pressurized state at any given time. In an alternate implementation, the pressure may be increased in each canister by operation of compression system 200. When inlet valves 122 and 124 are opened, pressure between canisters 302 and 304 will equalize, however, the equalized pressure in either canister may not be sufficient to inhibit air from entering the canisters during shutdown. In order to ensure that air is inhibited from entering the canisters, compression system 200 may be operated for a time sufficient to increase the pressure inside both canisters to a level at least greater than ambient pressure. Regardless of the method of pressurization of the canisters, once the canisters are pressurized, inlet valves 122 and 124 are closed, trapping the pressurized air inside the canisters, which inhibits air from entering the canisters during the shutdown period.

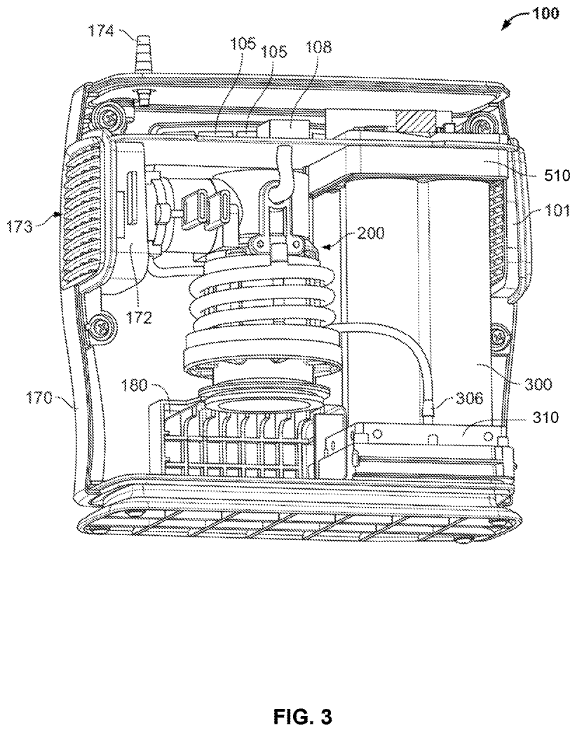

Referring to FIG. 3, an implementation of an oxygen concentrator 100 is depicted. Oxygen concentrator 100 includes a compression system 200, a canister system 300, and a power supply 180 disposed within an outer housing 170. Inlets 101 are located in outer housing 170 to allow air from the environment to enter oxygen concentrator 100. Inlets 101 may allow air to flow into the compartment to assist with cooling of the components in the compartment. Power supply 180 provides a source of power for the oxygen concentrator 100. Compression system 200 draws air in through the inlet 105 and muffler 108. Muffler 108 may reduce noise of air being drawn in by the compression system and also may include a desiccant material to remove water from the incoming air. Oxygen concentrator 100 may further include fan 172 used to vent air and other gases from the oxygen concentrator via outlet 173.

Compression System

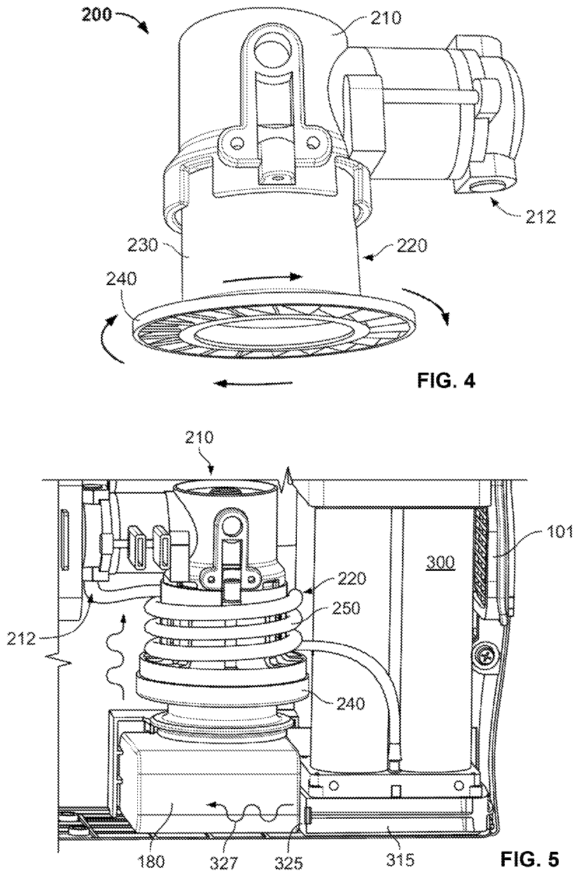

In some implementations, compression system 200 includes one or more compressors. In another implementation, compression system 200 includes a single compressor, coupled to all of the canisters of canister system 300. Turning to FIGS. 4 and 5, a compression system 200 is depicted that includes compressor 210 and motor 220. Motor 220 is coupled to compressor 210 and provides an operating force to the compressor to operate the compression mechanism. For example, motor 220 may be a motor providing a rotating component that causes cyclical motion of a component of the compressor that compresses air. When compressor 210 is a piston type compressor, motor 220 provides an operating force which causes the piston of compressor 210 to be reciprocated. Reciprocation of the piston causes compressed air to be produced by compressor 210. The pressure of the compressed air is, in part, estimated by the speed at which the compressor is operated (e.g., how fast the piston is reciprocated). Motor 220, therefore, may be a variable speed motor that is operable at various speeds to dynamically control the pressure of air produced by compressor 210.

In one implementation, compressor 210 includes a single head wobble type compressor having a piston. Other types of compressors may be used such as diaphragm compressors and other types of piston compressors. Motor 220 may be a DC or AC motor and provides the operating power to the compressing component of compressor 210. Motor 220, in an implementation, may be a brushless DC motor. Motor 220 may be a variable speed motor configured to operate the compressing component of compressor 210 at variable speeds. Motor 220 may be coupled to controller 400, as depicted in FIG. 2, which sends operating signals to the motor to control the operation of the motor. For example, controller 400 may send signals to motor 220 to: turn the motor on, turn the motor off, and set the operating speed of the motor. Thus, as illustrated in FIG. 2, the compression system 200 may include a speed sensor 201. The speed sensor may be a motor speed transducer used to determine a rotational velocity of the motor 220 and/or other reciprocating operation of the compression system 200. For example, a motor speed signal from the motor speed transducer may be provided to the controller 400. The speed sensor or motor speed transducer may, for example, be a Hall effect sensor. The controller 400 may operate the compression system 200 via the motor 220 based on the speed signal and/or any other sensor signal of the oxygen concentrator, such as a pressure sensor (e.g., accumulator pressure sensor 107). Thus, as illustrated in FIG. 2, the controller 400 receives sensor signals, such as a speed signal from the speed sensor 201 and accumulator pressure signal from the accumulator pressure sensor 107. With such signal(s), the controller may implement one or more control loops (e.g., feedback control) for operation of the compression system based on sensor signals such as accumulator pressure and/or motor speed as described in more detail herein.

Compression system 200 inherently creates substantial heat. Heat is caused by the consumption of power by motor 220 and the conversion of power into mechanical motion. Compressor 210 generates heat due to the increased resistance to movement of the compressor components by the air being compressed. Heat is also inherently generated due to adiabatic compression of the air by compressor 210. Thus, the continual pressurization of air produces heat in the enclosure. Additionally, power supply 180 may produce heat as power is supplied to compression system 200. Furthermore, users of the oxygen concentrator may operate the device in unconditioned environments (e.g., outdoors) at potentially higher ambient temperatures than indoors, thus the incoming air will already be in a heated state.

Heat produced inside oxygen concentrator 100 can be problematic. Lithium ion batteries are generally employed as power supplies for oxygen concentrators due to their long life and light weight. Lithium ion battery packs, however, are dangerous at elevated temperatures and safety controls are employed in oxygen concentrator 100 to shut down the system if dangerously high power supply temperatures are detected. Additionally, as the internal temperature of oxygen concentrator 100 increases, the amount of oxygen generated by the concentrator may decrease. This is due, in part, to the decreasing amount of oxygen in a given volume of air at higher temperatures. If the amount of produced oxygen drops below a predetermined amount, the oxygen concentrator 100 may automatically shut down.

Because of the compact nature of oxygen concentrators, dissipation of heat can be difficult. Solutions typically involve the use of one or more fans to create a flow of cooling air through the enclosure. Such solutions, however, require additional power from the power supply 180 and thus shorten the portable usage time of the oxygen concentrator. In an implementation, a passive cooling system may be used that takes advantage of the mechanical power produced by motor 220. Referring to FIGS. 4 and 5, compression system 200 includes motor 220 having an external rotating armature 230. Specifically, armature 230 of motor 220 (e.g. a DC motor) is wrapped around the stationary field that is driving the armature. Since motor 220 is a large contributor of heat to the overall system it is helpful to transfer heat off the motor and sweep it out of the enclosure. With the external high speed rotation, the relative velocity of the major component of the motor and the air in which it exists is very high. The surface area of the armature is larger if externally mounted than if it is internally mounted. Since the rate of heat exchange is proportional to the surface area and the square of the velocity, using a larger surface area armature mounted externally increases the ability of heat to be dissipated from motor 220. The gain in cooling efficiency by mounting the armature externally, allows the elimination of one or more cooling fans, thus reducing the weight and power consumption while maintaining the interior of the oxygen concentrator within the appropriate temperature range. Additionally, the rotation of the externally mounted armature creates movement of air proximate to the motor to create additional cooling.

Moreover, an external rotating armature may help the efficiency of the motor, allowing less heat to be generated. A motor having an external armature operates similar to the way a flywheel works in an internal combustion engine. When the motor is driving the compressor, the resistance to rotation is low at low pressures. When the pressure of the compressed air is higher, the resistance to rotation of the motor is higher. As a result, the motor does not maintain consistent ideal rotational stability, but instead surges and slows down depending on the pressure demands of the compressor. This tendency of the motor to surge and then slow down is inefficient and therefore generates heat. Use of an external armature adds greater angular momentum to the motor which helps to compensate for the variable resistance experienced by the motor. Since the motor does not have to work as hard, the heat produced by the motor may be reduced.

In an implementation, cooling efficiency may be further increased by coupling an air transfer device 240 to external rotating armature 230. In an implementation, air transfer device 240 is coupled to the external armature 230 such that rotation of the external armature 230 causes the air transfer device 240 to create an air flow that passes over at least a portion of the motor. In an implementation, the air transfer device 240 includes one or more fan blades coupled to the external armature 230. In an implementation, a plurality of fan blades may be arranged in an annular ring such that the air transfer device 240 acts as an impeller that is rotated by movement of the external rotating armature 230. As depicted in FIGS. 4 and 5, air transfer device 240 may be mounted to an outer surface of the external armature 230, in alignment with the motor 220. The mounting of the air transfer device 240 to the armature 230 allows air flow to be directed toward the main portion of the external rotating armature 230, providing a cooling effect during use. In an implementation, the air transfer device 240 directs air flow such that a majority of the external rotating armature 230 is in the air flow path.

Further, referring to FIGS. 4 and 5, air pressurized by compressor 210 exits compressor 210 at compressor outlet 212. A compressor outlet conduit 250 is coupled to compressor outlet 212 to transfer the compressed air to canister system 300. As noted previously, compression of air causes an increase in the temperature of the air. This increase in temperature can be detrimental to the efficiency of the oxygen concentrator. In order to reduce the temperature of the pressurized air, compressor outlet conduit 250 is placed in the air flow path produced by air transfer device 240. At least a portion of compressor outlet conduit 250 may be positioned proximate to motor 220. Thus, air flow, created by air transfer device 240, may contact both motor 220 and compressor outlet conduit 250. In one implementation, a majority of compressor outlet conduit 250 is positioned proximate to motor 220. In an implementation, the compressor outlet conduit 250 is coiled around motor 220, as depicted in FIG. 5.

In an implementation, the compressor outlet conduit 250 is composed of a heat exchange metal. Heat exchange metals include, but are not limited to, aluminum, carbon steel, stainless steel, titanium, copper, copper-nickel alloys or other alloys formed from combinations of these metals. Thus, compressor outlet conduit 250 can act as a heat exchanger to remove heat that is inherently caused by compression of the air. By removing heat from the compressed air, the number of molecules in a given volume at a given pressure is increased. As a result, the amount of oxygen enriched air that can be generated by each canister during each pressure swing cycle may be increased.

The heat dissipation mechanisms described herein are either passive or make use of elements required for the oxygen concentrator 100. Thus, for example, dissipation of heat may be increased without using systems that require additional power. By not requiring additional power, the run-time of the battery packs may be increased and the size and weight of the oxygen concentrator may be minimized. Likewise, use of an additional box fan or cooling unit may be eliminated Eliminating such additional features reduces the weight and power consumption of the oxygen concentrator.

As discussed above, adiabatic compression of air causes the air temperature to increase. During venting of a canister in canister system 300, the pressure of the gas being released from the canisters decreases. The adiabatic decompression of the gas in the canister causes the temperature of the gas to drop as it is vented. In an implementation, the cooled vented gases 327 from canister system 300 are directed toward power supply 180 and toward compression system 200. In an implementation, base 315 of canister system 300 receives the vented gases from the canisters. The vented gases 327 are directed through base 315 toward outlet 325 of the base 315 and toward power supply 180. The vented gases, as noted, are cooled due to decompression of the gases and therefore passively provide cooling to the power supply. When the compression system 200 is operated, the air transfer device 240 will gather the cooled vented gases and direct the gases toward the motor 220 of compression system 200. Fan 172 may also assist in directing the vented gas across compression system 200 and out of the housing 170. In this manner, additional cooling may be obtained without requiring any further power requirements from the battery.

### Canister System

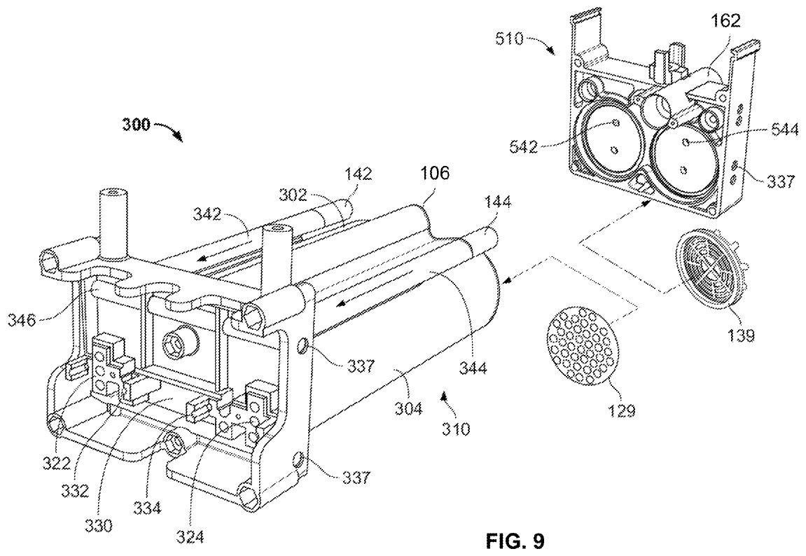

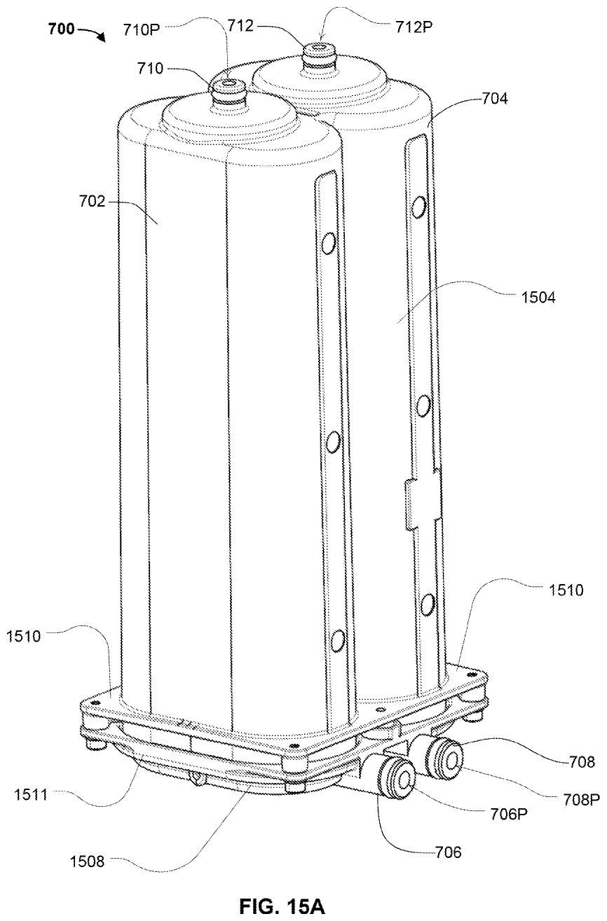

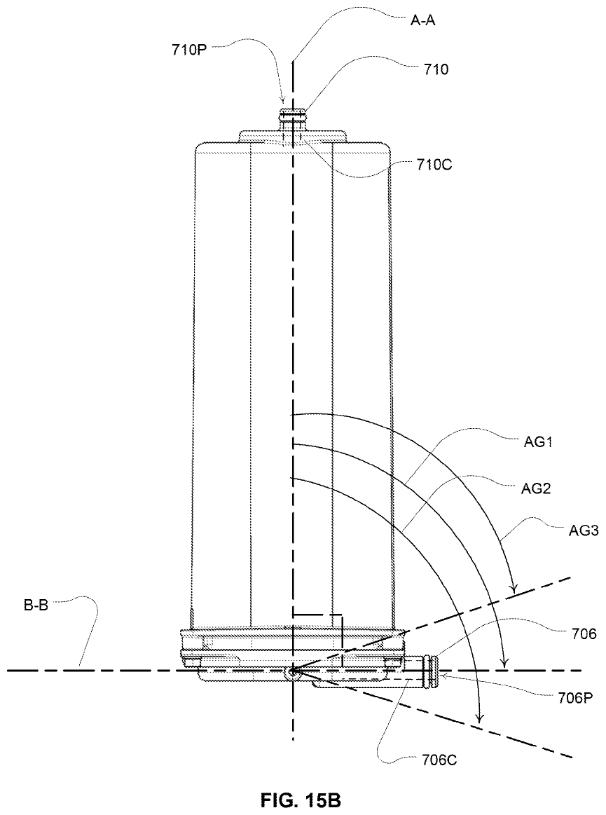

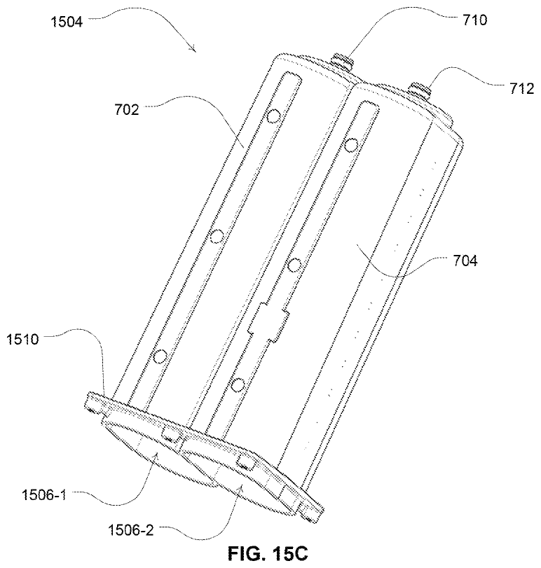

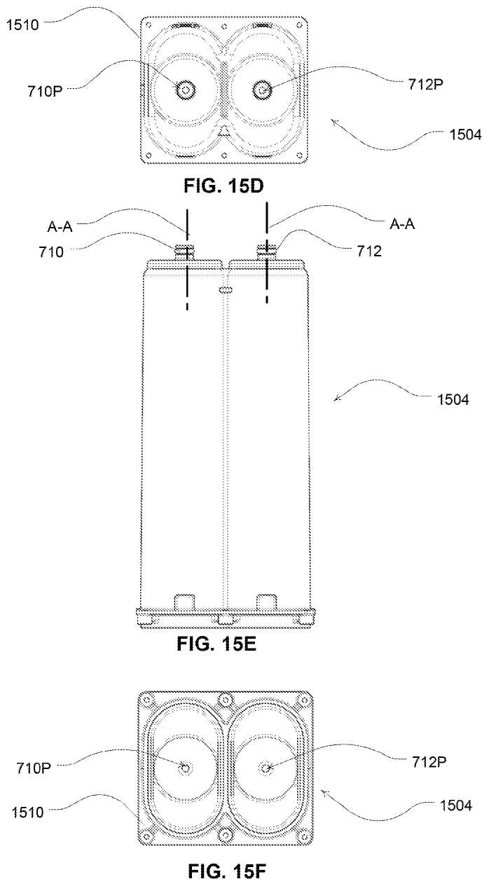

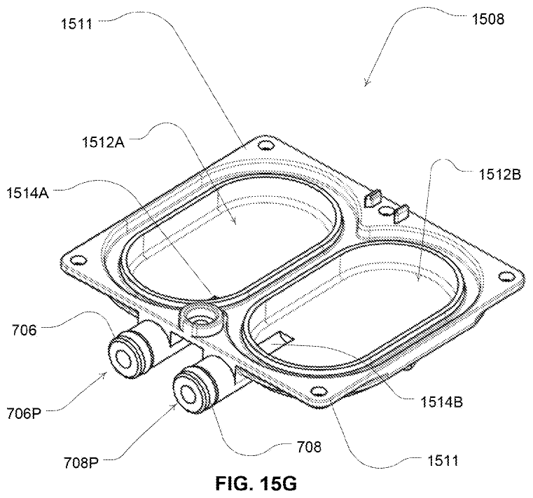

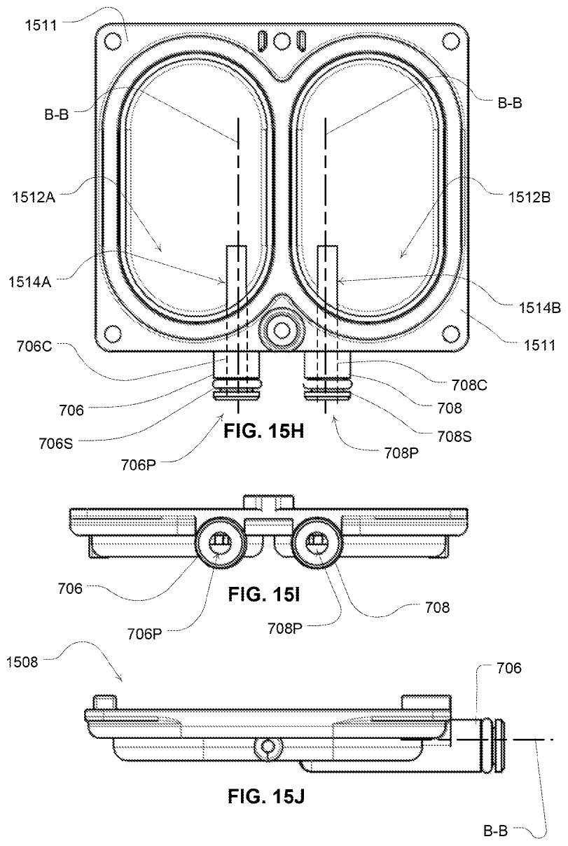

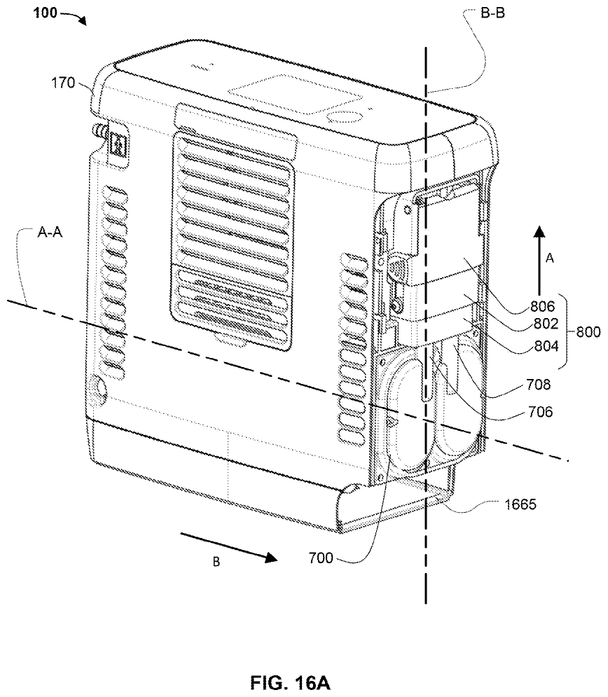

Oxygen concentrator 100 may include at least two canisters, each canister including a gas separation adsorbent. Examples may be considered in relation to the version shown in FIGS. 9-13, which show a canister assembly that is generally integrated into a housing of a POC and typically requires a service technician and tools to install and remove. An alternative version is shown in FIGS. 15A-15J as a removable canister assembly that may be easily inserted and removed from the POC.

The canisters of oxygen concentrator 100 may be disposed in or formed from a molded housing. In an implementation, canister system 300 includes two housing components 310 and 510, as depicted in FIG. 9. In various implementations, the housing components 310 and 510 of the oxygen concentrator 100 may form a two-part molded plastic frame that defines two canisters 302 and 304 and accumulator 106. The housing components 310 and 510 may be formed separately and then coupled together. In some implementations, housing components 310 and 510 may be injection molded or compression molded. Housing components 310 and 510 may be made from a thermoplastic polymer such as polycarbonate, methylene carbide, polystyrene, acrylonitrile butadiene styrene (ABS), polypropylene, polyethylene, or polyvinyl chloride. In another implementation, housing components 310 and 510 may be made of a thermoset plastic or metal (such as stainless steel or a lightweight aluminum alloy). Lightweight materials may be used to reduce the weight of the oxygen concentrator 100. In some implementations, the two housings 310 and 510 may be fastened together using screws or bolts. Alternatively, housing components 310 and 510 may be solvent welded together. Installation of the canister assembly 300 of FIG. 9 into or out of the housing of POC shown in FIG. 1 generally requires removal of the outer housing 170 of the POC 100 and the use of tools, such that its replacement is typically performed by technician.

As shown in FIGS. 9-13, valve seats 322, 324, 332, and 334 and air pathways of conduit 330 and 346 may be integrated into the housing component 310 to reduce the number of sealed connections needed throughout the air flow of the oxygen concentrator 100.

Air pathways/tubing between different sections in housing components 310 and 510 may take the form of molded conduits. Conduits in the form of molded channels for air pathways may occupy multiple planes in housing components 310 and 510. For example, the molded air conduits may be formed at different depths and at different x,y,z positions in housing components 310 and 510. In some implementations, a majority or substantially all of the conduits may be integrated into the housing components 310 and 510 to reduce potential leak points.

In some implementations, prior to coupling housing components 310 and 510 together, O-rings may be placed between various points of housing components 310 and 510 to ensure that the housing components are properly sealed. In some implementations, components may be integrated and/or coupled separately to housing components 310 and 510. For example, tubing, flow restrictors (e.g., press fit flow restrictors), oxygen sensors, gas separation adsorbents, check valves, plugs, processors, power supplies, etc. may be coupled to housing components 310 and 510 before and/or after the housing components are coupled together.

In some implementations, apertures 337 leading to the exterior of housing components 310 and 510 may be used to insert devices such as flow restrictors. Apertures may also be used for increased moldability. One or more of the apertures may be plugged after molding (e.g., with a plastic plug). In some implementations, flow restrictors may be inserted into passages prior to inserting plugs to seal the passages. Press fit flow restrictors may have diameters that may allow a friction fit between the press fit flow restrictors and their respective apertures. In some implementations, an adhesive may be added to the exterior of the press fit flow restrictors to hold the press fit flow restrictors in place once inserted. In some implementations, the plugs may have a friction fit with their respective tubes (or may have an adhesive applied to their outer surface). The press fit flow restrictors and/or other components may be inserted and pressed into their respective apertures using a narrow tip tool or rod (e.g., with a diameter less than the diameter of the respective aperture). In some implementations, the press fit flow restrictors may be inserted into their respective tubes until they abut a feature in the tube to halt their insertion. For example, the feature may include a reduction in radius. Other features are also contemplated (e.g., a bump in the side of the tubing, threads, etc.). In some implementations, press fit flow restrictors may be molded into the housing components (e.g., as narrow tube segments).

In some implementations, spring baffle 139 may be placed into respective canister receiving portions of housing components 310 and 510 with the spring side of the baffle 139 facing the exit of the canister. Spring baffle 139 may apply force to gas separation adsorbent in the canister while also assisting in preventing gas separation adsorbent from entering the exit apertures. Use of a spring baffle 139 may keep the gas separation adsorbent compact while also allowing for expansion (e.g., thermal expansion). Keeping the gas separation adsorbent compact may prevent the gas separation adsorbent from breaking during movement of the oxygen concentrator 100.

In some implementations, filter 129 may be placed into respective canister receiving portions of housing components 310 and 510 facing the inlet of the respective canisters. The filter 129 removes particles from the feed gas stream entering the canisters.

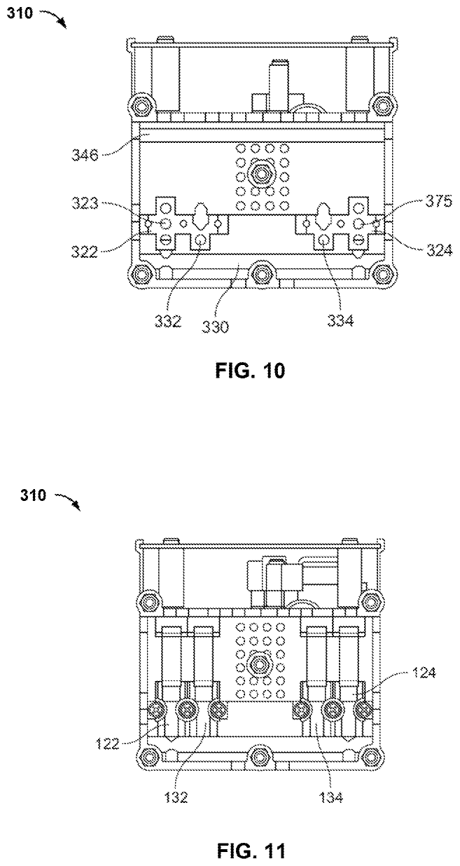

In some implementations, pressurized air from the compression system 200 may enter air inlet 306. Air inlet 306 is coupled to inlet conduit 330. Air enters housing component 310 through inlet 306 and travels through conduit 330, and then to valve seats 322 and 324. FIG. 10 and FIG. 11 depict an end view of housing 310. FIG. 10 depicts an end view of housing 310 prior to fitting valves to housing 310. FIG. 11 depicts an end view of housing 310 with the valves fitted to the housing 310. Valve seats 322 and 324 are configured to receive inlet valves 122 and 124 respectively. Inlet valve 122 is coupled to canister 302 and inlet valve 124 is coupled to canister 304. Housing 310 also includes valve seats 332 and 334 configured to receive outlet valves 132 and 134 respectively. Outlet valve 132 is coupled to canister 302 and outlet valve 134 is coupled to canister 304. Inlet valves 122/124 are used to control the passage of air from conduit 330 to the respective canisters.

In an implementation, pressurized air is sent into one of canisters 302 or 304 while the other canister is being vented. For example, during use, inlet valve 122 is opened while inlet valve 124 is closed. Pressurized air from compression system 200 is forced into canister 302, while being inhibited from entering canister 304 by inlet valve 124. During pressurization of canister 302, outlet valve 132 is closed and outlet valve 134 is opened. Similar to the inlet valves, outlet valves 132 and 134 are operated out of phase with each other. Valve seat 322 includes an opening 323 that passes through housing 310 into canister 302. Similarly valve seat 324 includes an opening 375 that passes through housing 310 into canister 302. Air from conduit 330 passes through openings 323 or 375 if the respective valves 322 and 324 are open, and enters a canister.

Check valves 142 and 144 (See FIG. 9) are coupled to canisters 302 and 304, respectively. Check valves 142 and 144 are one way valves that may be passively operated by the pressure differentials that occur as the canisters are pressurized and vented. Oxygen enriched air produced in canisters 302 and 304 passes from the canisters into openings 542 and 544 of housing component 510. A passage (not shown) links openings 542 and 544 to conduits 342 and 344, respectively. Oxygen enriched air produced in canister 302 passes from the canister though opening 542 and into conduit 342 when the pressure in the canister is sufficient to open check valve 142. When check valve 142 is open, oxygen enriched air flows through conduit 342 toward the end of housing 310. Similarly, oxygen enriched air produced in canister 304 passes from the canister through opening 544 and into conduit 344 when the pressure in the canister is sufficient to open check valve 144. When check valve 144 is open, oxygen enriched air flows through conduit 344 toward the end of housing 310.

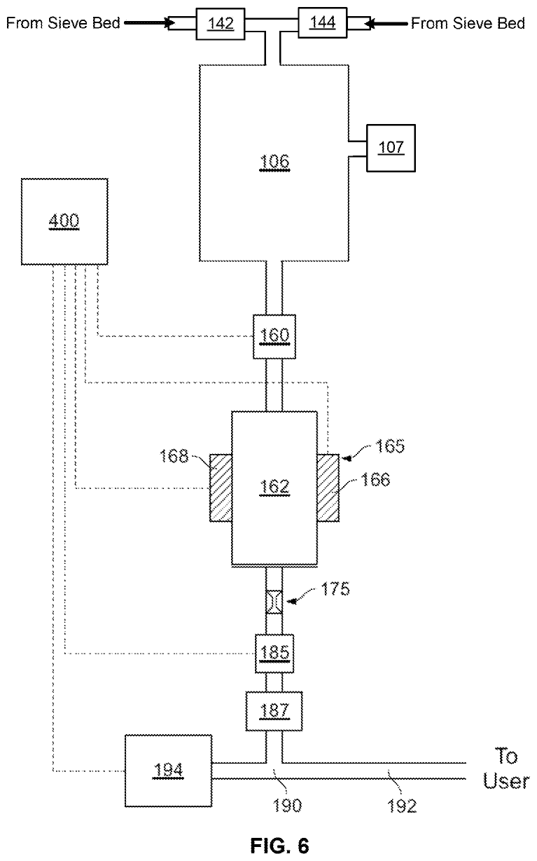

Oxygen enriched air from either canister travels through conduit 342 or 344 and enters conduit 346 formed in housing 310. Conduit 346 includes openings that couple the conduit to conduit 342, conduit 344 and accumulator 106. Thus, oxygen enriched air, produced in canister 302 or 304, travels to conduit 346 and passes into accumulator 106. As illustrated in FIG. 2, gas pressure within the accumulator 106 may be measured by a sensor, such as with an accumulator pressure sensor 107. (See also FIG. 6.) Thus, the accumulator pressure sensor provides a signal representing the pressure of the accumulated oxygen enriched air. An example of a suitable pressure transducer is a sensor from the HONEYWELL ASDX series. An alternative suitable pressure transducer is a sensor from the NPA Series from GENERAL ELECTRIC. In some implementations, the pressure sensor may alternatively measure pressure of the gas outside of the accumulator 106, such as in an output path between the accumulator 106 and a valve (e.g., supply valve 160) that gates the release of the oxygen enriched air for delivery to a user in a bolus.

After some time, the gas separation adsorbent will become saturated with nitrogen and will be unable to separate significant amounts of nitrogen from incoming air. When the gas separation adsorbent in a canister reaches this saturation point, the inflow of compressed air is stopped and the canister is vented to remove nitrogen. Canister 302 is vented by closing inlet valve 122 and opening outlet valve 132. Outlet valve 132 releases the vented gas from canister 302 into the volume defined by the end of housing 310. Foam material may cover the end of housing 310 to reduce the sound made by release of gases from the canisters. Similarly, canister 304 is vented by closing inlet valve 124 and opening outlet valve 134. Outlet valve 134 releases the vented gas from canister 304 into the volume defined by the end of housing 310.

While canister 302 is being vented, canister 304 is pressurized to produce oxygen enriched air in the same manner described above. Pressurization of canister 304 is achieved by closing outlet valve 134 and opening inlet valve 124. The oxygen enriched air exits canister 304 through check valve 144.

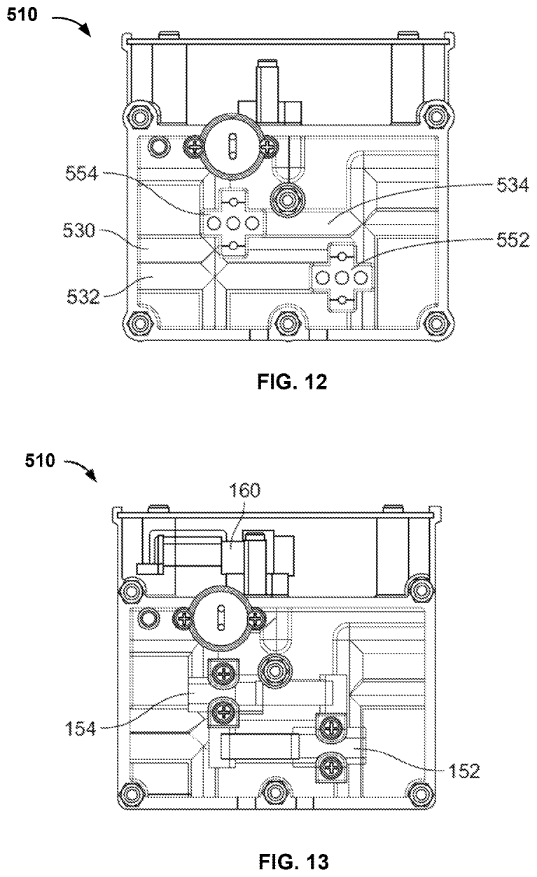

In an exemplary implementation, a portion of the oxygen enriched air may be transferred from canister 302 to canister 304 when canister 304 is being vented of nitrogen. Transfer of oxygen enriched air from canister 302 to canister 304, during venting of canister 304, helps to further purge nitrogen (and other gases) from the canister. Flow of oxygen enriched air between the canisters is controlled using flow restrictors and valves, as depicted in FIG. 2. Three conduits are formed in housing component 510 for use in transferring oxygen enriched air between canisters. As shown in FIG. 12, conduit 530 couples canister 302 to canister 304. Flow restrictor 151 (not shown) is disposed in conduit 530, between canister 302 and canister 304 to restrict flow of oxygen enriched air during use. Conduit 532 also couples canister 302 to 304. Conduit 532 is coupled to valve seat 552 which receives valve 152, as shown in FIG. 13. Flow restrictor 153 (not shown) is disposed in conduit 532, between canister 302 and 304. Conduit 534 also couples canister 302 to 304. Conduit 534 is coupled to valve seat 554 which receives valve 154, as shown in FIG. 13. Flow restrictor 155 (not shown) is disposed in conduit 534, between canister 302 and 304. The pair of equalization/vent valves 152/154 work with flow restrictors 153 and 155 to optimize the air flow balance between the two canisters.

Oxygen enriched air in accumulator 106 passes through supply valve 160 into expansion chamber 162 which is formed in housing component 510. An opening (not shown) in housing component 510 couples accumulator 106 to supply valve 160. In an implementation, expansion chamber 162 may include one or more devices configured to estimate an oxygen concentration of gas passing through the chamber.

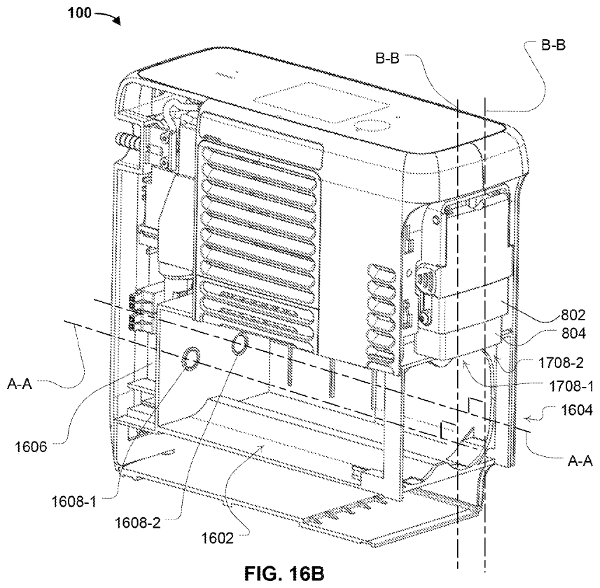

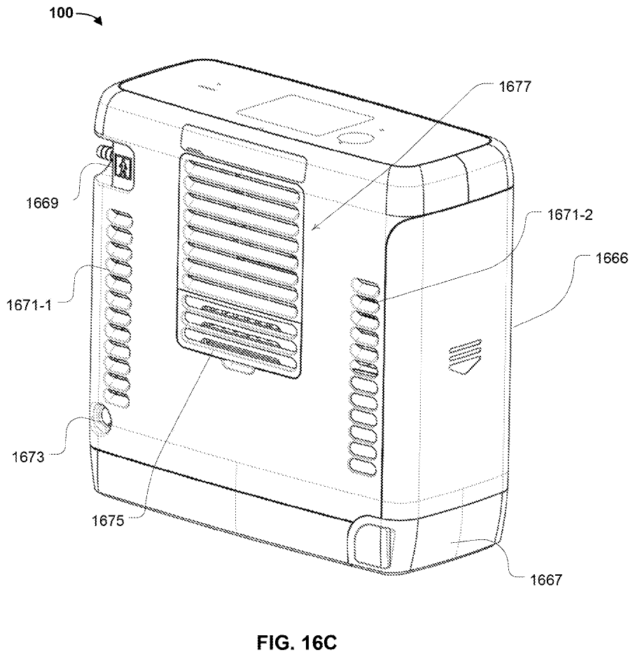

### Removable Canister Assembly (FIGS. 15-16)

As previously mentioned, in some implementations, to facilitate removability by its user, the canisters of oxygen concentrator 100 may be formed as shown in FIGS. 15-16. In relation to pressurization and depressurization operations, such canisters are similar to the canisters described in relation to FIGS. 9-13 but are otherwise structured to facilitate easy replacement and removal from the POC. As illustrated in the example of FIG. 15A, sieve bed canister assembly 700 includes canisters 702 and 704. Each canister provides a separately pressurizable container for gas separation adsorbent that is comparable to the sieve bed previously described. As illustrated in FIGS. 15C to 15F, the canister assembly 700 may have a container portion 1504, which may define one or more container volumes 1506-1, 1506-2 for containing gas separation adsorbent of the sieve bed(s). The canister assembly 700 may also include one or more cap portions 1508. The container portion 1504 may include one or more mounting flanges 1510 for mounting or joining the cap portion(s) 1508 onto the container portion(s) 1504 to form each or both canisters of the canister assembly 700. Thus, the cap portion 1508 may similarly include a flange portion 1511 corresponding with the flanges 1510 such that they may optionally be joined by various joining means (e.g., welding or fasteners such as screws, bolts or rivets).

Each canister 702, 704 includes an inlet (or air inlet) and an outlet (or air outlet). For example, as shown in the example of FIG. 15A, a first canister (canister 702) includes an inlet 706 and an outlet 710 that provide pneumatic access to the gas separation adsorbent of the sieve bed formed by the first canister. A second canister (canister 704) includes an inlet 708 and an outlet 712 that provide pneumatic access to the gas separation adsorbent of the sieve bed formed by the second canister. As illustrated, any or all of the inlets and/or outlets may be formed as projections or nipples for respective insertion within a coupler or port/orifice of a sieve bed compartment of the POC when inserted into the POC outer housing for use. Such inlets and outlets may have a channel (e.g., cylindrical) within each so that each serves as a path for the gas transfer involved in pressure swing and/or vacuum swing operations of the respective sieve bed of the canister. Such an inlet channel 706C for inlet 706 and an outlet channel 710C for outlet 710 are illustrated in FIG. 15B. Comparable channels may exist for the other inlet and outlet of the canister assembly 700. In the illustrated example of FIG. 15A, each outlet is part of a container portion 1504 and each inlet is part of a cap portion 1508. As seen in FIGS. 15G and 15H, a discrete recess 1512A, 1512B in the cap portion 1508 for each canister may be generally planar or have a generally planar surface for covering an end of the volume of the container portion 1504 where the adsorbent of the sieve bed resides. The discrete recess 1512A, 1512B may include an inlet groove 1514A, 1514B leading to channel 706C, 708C of the inlets 706, 708 respectively. Moreover, each inlet may have an inlet seal 706S, 708S respectively, such as a flexible rubber O-ring to create a sealed connection with the oxygen concentrator 100 for pressurized operations. The outlets 710, 712 may similarly include outlet seals.

As illustrated in FIGS. 15A and 15B, the opening (or port) of the inlets and outlets may be positioned at opposing ends of the canister assembly 700. For example, the outlet 710 at its port 710P may be proximate to or at a first end (or top end) of the canister 702 whereas inlet 706 at its port 706P may be proximate to or at a second end (or bottom end) of the canister 702. Similarly, the outlet 712 at its port 712P may be proximate to or at a first end or top end of the canister 704 whereas inlet 708 at its port 708P may be proximate to or at a second end (or bottom end) of the canister 704. While the terms “inlet” and “outlet” are generally used herein to assist with an explanation of features of the canister, such terms are not intended to require only a single direction of gas transfer since it will be recognized that the PSA or VSA process can involve ingress and egress of gas at a common end of the canister depending on the cycle of the process. However, in the example of FIG. 15A, the POC may be configured so that the outlets are associated with an output of a product gas (e.g., oxygen enriched air) whereas the inlets may be associated with an introduction of ambient gas (e.g., air) to the sieve bed for the adsorption process. Nevertheless, it will be understood that such functions of these inlets and outlets may be reversed depending on the implementation of the controlled flow path of the POC (i.e., operation of its valves and manifolds) in which the canister assembly 700 is inserted.

As illustrated in more detail in FIG. 15B, the inlet and outlet of each canister may be formed with a particular angle relative to each other to promote removable insertion and/or securing of the canister assembly 700 within the compartment of the POC for use. For example, the inlets may each be understood to have an imaginary axis (or central axis), such as a central axis that extends centrally along a path or channel of the inlet through its port, or such an axis that is central to an outer structure of the inlet such as its cylindrical or nipple structure. Such a central axis is illustrated in FIG. 15B with line B-B. Such an axis illustrated with line B-B may be generally parallel with the generally planar surface of the cap portion 1508. Similarly, the outlets may each be understood to have an imaginary axis (or central axis), such as a central axis that extends centrally along a path or channel of the outlet through its port or such an axis that is central to an outer structure of the outlet such as its cylindrical or nipple structure. Such a central axis is illustrated in FIG. 15B with line A-A. Such an axis illustrated with line A-A may be generally perpendicular with the generally planar surface of the cap portion 1508. In addition, the line A-A may be generally parallel with the generally planar surface of the container portion 1504. Moreover, the axes associated with lines A-A of the outlets may be generally (or approximately or substantially) parallel to each other. For instance and as illustrated in FIG. 15E, the line A-A of the outlet 710 is substantially parallel to the line A-A of the outlet 712. Similarly, the axes associated with lines B-B of the inlets may be generally (or approximately or substantially) parallel with each other. For instance, and as illustrated in FIG. 15H, the line B-B of the inlet 706 is substantially parallel to the line B-B of the inlet 708. Furthermore, an angle formed by such axes (e.g., such as those illustrated by lines A-A and B-B corresponding to an outlet and inlet respectively of a given canister) of the canister assembly 700 may be an acute angle, an obtuse angle, a right angle, or an angle within a particular range such as to not be substantially parallel or antiparallel. For example, such an angle (see example angles AG1, AG2 and AG3 in FIG. 15B) may be in a range of forty (40) degrees and one hundred thirty-five (135) degrees or other subrange therein (e.g., 50 degrees to 125 degrees). For example, the angle formed by such axes of a canister of the canister assembly 700 may be approximately ninety (90) degrees such that the central axes (or lines) A-A and B-B are substantially perpendicular to each other. In other words, the inlet is configured to be substantially orthogonal to the outlet of a given canister. As discussed in more detail herein, such angling may permit the coupling of the inlets or outlets of the canister assembly with respective manifolds of the POC and may promote removable insertion and/or securing of the canister assembly 700 within the POC. In this regard, as discussed in more detail herein, the manifolds of the POC may be configured to receive (or couple with) the inlets and outlets of the canister assembly 700 and as such, the manifolds, as discussed in more detail herein in reference to FIGS. 16A and 16B, may similarly have axes that form an angle similar to those of the canister. In this regard, the axes of the outlets of the canister assembly may align with axes of ports of an outlet manifold such that these axes constitute a common direction of translation associated with coupling or insertion between the canister assembly and outlet manifold. Similarly, axes of the inlets of the canister assembly may align with axes of ports of an inlet manifold such that the axes constitute a common direction of translation associated with coupling or insertion between the canister assembly and the inlet manifold.

In some implementations, canisters 702 and 704 may be formed as a unitary structure, such as with a single container portion 1504 having discrete volumes for containing adsorbent of two sieve beds. In some implementations, canisters 702 and 704 may be formed as separate (or distinct) structures, such as with separate container portions 1504, that may optionally be joined by one or more mounting flanges 1510 for mounting or joining with the cap portion 1508. Thus, canisters 702 and 704 may be held together by one or more additional pieces or fasteners such that canister assembly 700 can be installed and removed as a single unit. For example, if canisters 702 and 704 are formed as separate structures, the cap portion 1508 containing the inlets (or air inlets) 706 and 708 may be attached to the bottom end of canisters 702 and 704 so that canister assembly 700 can then be handled as a single unit.

The canister assembly 700 may be understood to have a smaller deadspace when compared to other designs of removable canister assembly such as those where all inlet and outlet ports are at the same end of the removable canister assembly. This is because there is no need for any tube to convey the oxygen enriched air from the product end back to the feed end of each sieve bed, or the feed gas from the product end to the feed end.

As previously mentioned, the removable canister assembly 700 may be easily inserted and removed from the POC. This is illustrated by FIG. 16A, which shows canister assembly 700 installed into a compartment 1602 (see FIG. 16B) of oxygen concentrator 100, within the outer housing 170. The compartment 1602 is adapted to contain the canister assembly 700. As shown in the cutout view of FIG. 16B, compartment 1602 may be accessed by removing a portion of the outer housing 170, such as a lid (or canister cover or canister panel), for removing and/or inserting of the canister assembly 700 via a portal 1604, such as at a side, of the outer housing 170 of the oxygen concentrator 100. Such a lid 1666 is shown in FIG. 16C. Thus, insertion or removal of the canister assembly 700 may be achieved without disassembling or removing the entire outer housing 170 but may be achieved by removing the lid (shown in FIG. 16C) of the outer housing 170. As illustrated in FIGS. 16A and 16B, such insertion of the canister assembly 700 may involve engaging the ports of the inlets and/or outlets of the canister assembly 700 with a coupling of one or more manifolds adjacent to the compartment 1602 and within the outer housing 170 of the oxygen concentrator 100. Each outlet of the canister assembly 700 may have a nipple for joining to a coupling of a manifold. For example, as illustrated in FIG. 16B, the outlets of the canister assembly 700 may be coupled to manifold 1606 (e.g., an outlet manifold) at the couplings 1608-1, 1608-2 (e.g., outlet couplings) of the manifold 1606, which may permit pneumatic sealing of the outlets of the canister assembly 700. Such couplings (e.g., outlet couplings 1608-1, 1608-2) may have a pneumatically sealable structure to complement a reciprocal structure (or complementary structure) of the outlet of the canister assembly 700. For example, such couplings (e.g., outlet couplings 1608-1, 1608-2) may be configured as orifices to receive nipples of the outlets of the canister assembly 700 within channels of the orifices. Moreover, each of these couplings (e.g., outlet couplings 1608-1, 1608-2) may have a central axis, such as along its orifice or the channel thereof, that complements (or aligns with or is parallel to) the central axis of a respective outlet of the canister assembly 700 as previously described (see lines A-A of FIG. 16B). As such, the outlets of the canister assembly 700 may be aligned with or along the same axes as the orifices (or channels) of the couplings of the manifold 1606. Consequently, movement of the canister assembly 700 within the compartment 1602 may be controlled by the complementary shaped structures of the compartment and canister assembly, such that canister assembly 700 moves along the central axis of the couplings (e.g., outlet couplings 1608-1, 1608-2) of the manifold 1606. In other words, the canister assembly 700 may slide in and out of the compartment 1602 along the axis A-A. The manifold 1606 may be generally affixed within the oxygen concentrator 100 as a stationary component. The manifold 1606 may also include one or more valves, such as any of valves (or control valves) 152, 154 when the manifold 1606 is an outlet manifold, or valves 122, 132, 124, 134 if the manifold 1606 is an inlet manifold.

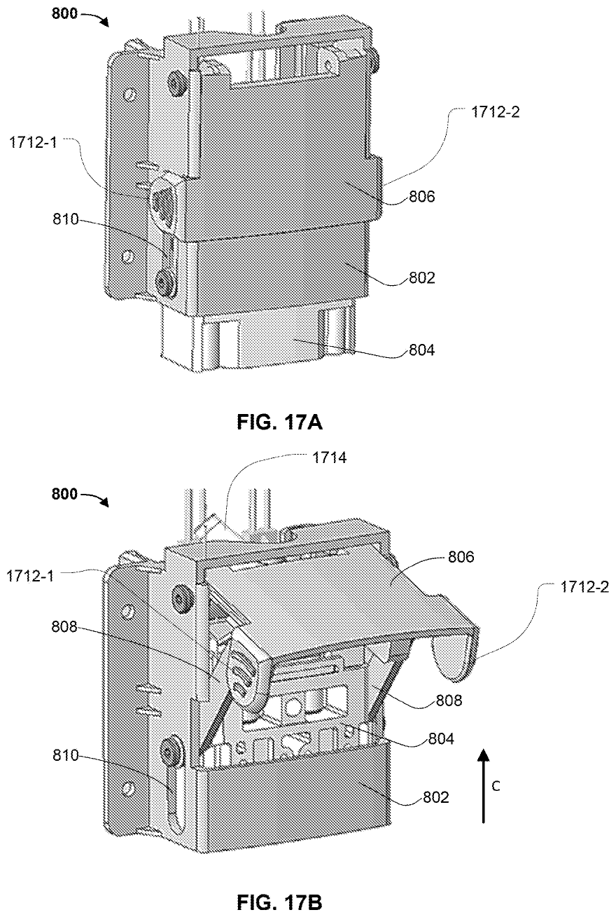

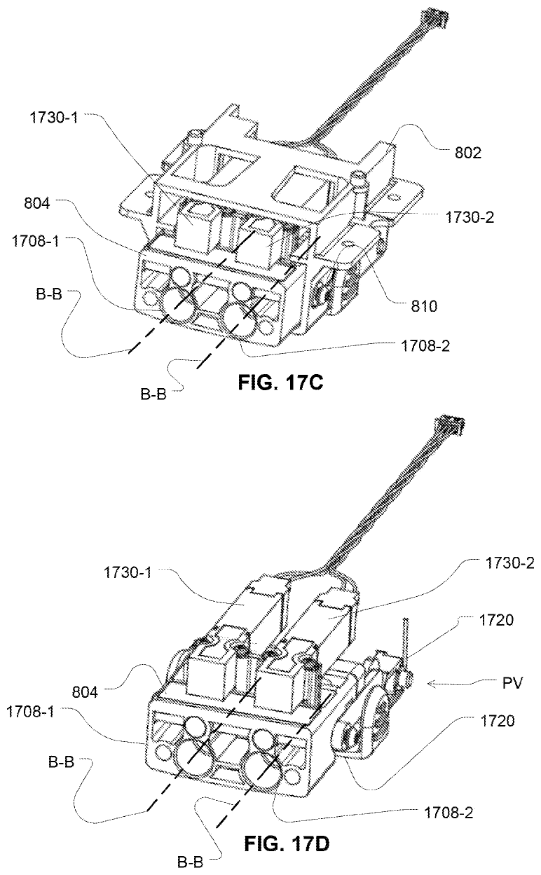

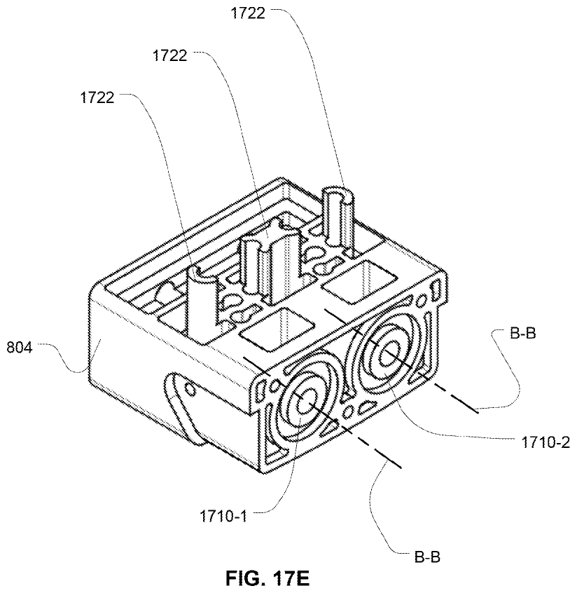

Similarly, as illustrated in FIGS. 16A and 16B, the inlets of the canister assembly 700 may be coupled to manifold 804 (e.g., an inlet manifold) with additional couplings 1708-1, 1708-2 (e.g., inlet couplings) (best seen in FIG. 17C), which may permit pneumatic sealing of the inlets of the canister assembly 700. Such couplings (e.g., outlet couplings 1708-1, 1708-2) may have a suitable pneumatically sealable structure to complement a reciprocal structure (or complementary structure) of the inlet of the canister assembly 700. For example, such couplings (e.g., outlet couplings 1708-1, 1708-2) may be configured as orifices to receive nipples of the inlets of the canister assembly 700 within channels of the orifices. Moreover, each of these couplings 1708-1, 1708-2 may have a central axis, such as along its insertion orifice or the channel thereof, that complements (or aligns with or is parallel to) the central axis of a respective inlet of the canister assembly 700 as previously described (see lines B-B of FIG. 16B). As such, the inlets of the canister assembly 700 may be aligned with or along the same axes as the orifices (or channels) of the couplings of the manifold 804. The manifold 804 may be generally affixed within the oxygen concentrator 100 as a traversing or moveable component as discussed in more detail herein. As shown in more detail in FIGS. 17C and 17D, the moveable manifold 804 may also include one or more valves 1730-1, 1730-2, such as any of control valves 122, 132, 124, 134 when the manifold 804 is an inlet manifold, or valves 152, 154 if the manifold 804 is an outlet manifold. Alternatively, the movable manifold 804 may comprise two valves (not shown) instead of four valves. Each of the two valves may be a 3/2 (three-way/two position valve) or a 5/2 valve (five-way/two position valve). As such, a smaller number of valves may be required to achieve the same effect. Such valves provide gating of the pneumatic flow through passages of the manifold as previously described for achieving the adsorption cycles with the respective sieve beds corresponding with the canisters 702, 704. Since such valves may be affixed with the manifold 804, the valve(s) move with the manifold 804. As illustrated in FIG. 17E, the example manifold 804 includes valve mounting brackets 1722 for affixing the valves to the manifold 804. The example manifold 804 also includes a first port 1710-1 and a second port 1710-2. The first port 1710-1 port and the second port 1710-2 are connected to the valves 1730-1 and 1730-2. As illustrated in FIG. 17B, the example manifold 804 also includes a barb 1714. The barb 1714 is connected to the first port 1710-1 and configured for connection to a flexible tube (not shown).

The oxygen concentrator 100 of the example of FIGS. 16A, 16B, and 16C is similar to those described above. As illustrated in FIG. 16A, the oxygen concentrator may have a battery compartment 1665 within the outer housing, separate from, and beneath the removable canister assembly 700. An optional battery compartment lid 1667 may be removable for accessing the battery. The outer housing 170 may have an indicator 1669 to indicate to a user that the adjacent conduit is for attachment to an airway delivery device, e.g. a cannula for receiving enriched air. The outer housing may also include a first and second sets of cooling system outlets 1671-1 and 1671-2. The outer housing may also include a charging port 1673 for supplying power to the oxygen concentrator for operations and/or for charging the battery. The outer housing may also have a removable panel 1675, such as with vent apertures. The removable panel 1675 may serve as an air filter and a single opening for feed gas to go in and move to the compressor inlet. The outer housing may have a set of cooling system inlets 1677.

Moreover, as shown in FIGS. 16A and 16B, the oxygen concentrator may also include a securing mechanism 800 that may permit removably engaging of the air inlets 706 and 708. The securing mechanism 800 may also secure canister assembly 700 within oxygen concentrator 100. Advantageously, the canister assembly 700 may be secured in position during operation of the oxygen concentrator 100, such as at a relatively high pressure. The securing mechanism 800 is configured such that it is movable between a closed position and an open position. Securing mechanism 800 is further shown in closed and open positions in FIGS. 17A and 17B, respectively. Thus, operation of the securing mechanism 800 may be utilized to achieve securement of the canister assembly 700 as well as pneumatic sealing with the canister assembly 700, such as when in operation. Securing mechanism 800 may be manipulated by a user for easy removal and insertion of the canister assembly 700.

The securing mechanism 800 may be configured such that movement of the securing mechanism 800 between the closed and open positions in a rotational manner (i.e. rotary motion) leads to linear motion of the manifold 804 (or inlet manifold 804). The linear motion of the manifold 804 may be along the axis B-B (or in a direction parallel to axis B-B). For example, as shown in FIGS. 17A and 17B, securing mechanism 800 may include guide frame 802, manifold 804, lever 806, linkage arms 808, and slots 810. In addition, the lever 806 of the securing mechanism 800 may include one or more grip handles 1712-1, 1712-2. Each grip handle 1712-1, 1712-2 may comprise a plurality of projections for enhancing frictional contact between the grip handle 1712-1, 1712-2 and the user's finger. The plurality of projections may be arranged on an outer surface of the grip handle 1712-1, 1712-2, thereby increasing finger grip.

When the lever 806 is in a closed position (which corresponds to the closed position of the securing mechanism 800) as shown in FIGS. 16A, 16B and 17A, manifold 804 protrudes or extends from guide frame 802. When the lever 806 is in an open position (which corresponds to the open position of the securing mechanism 800), as shown in FIG. 17B, manifold 804 may be retracted into guide frame 802. Operation of the lever 806, such as toward the open position from the closed position, may then move the manifold 804 in the direction of arrow C, thereby retracting the manifold 804 into and moving it within the guide frame 802. Operation of the lever 806, such as toward the closed position from the open position, may then move the manifold 804 in the opposite direction of arrow C, thereby extending the manifold 804 out of the guide frame 802 towards the inlets of the canister assembly 700. Consequently, the canister assembly 700 may be secured within oxygen concentrator 100 (e.g., a locked in position). Advantageously, the canister assembly 700 may be maintained in position during operation of the oxygen concentrator 100, such as at a relatively high pressure, because of the securing mechanism 800.

By opening and closing lever 806, force is applied through linkage arms 808 to manifold 804. Advantageously, lever 806 and linkage arms 808 provide a mechanical advantage by reducing or minimizing the amount of force needed to lock the canister assembly 700 in position. This is because the lever 806 amplifies the force from the user, and transmits the force through linkage arms 808 to lock the manifold 804 in the closed position and prevent the moving manifold 804 from disengaging from the canister assembly 700 during operation due to high pressure. Linkage arms 808 are pivotably coupled to lever 806, as well as manifold 804. Linkage arms 808 further engage with slots 810 (only one of which is visible in FIGS. 17B and 17C), such that opening and closing of lever 806 causes translational movement of manifold 804. For instance, opening and closing of lever 806 causes movement of manifold 804 in a direction parallel to line B-B. The linkage arms 808 may engage with slots 810 in any suitable way, including flanges at the end of each linkage arm 808, or through one or more bearings, screws, pins, etc. Likewise, linkage arms 808 may be pivotably coupled to lever 806 and manifold 804 in any suitable way, including through one or more screws, pins, axles, etc.

Optionally and as shown in FIG. 17D, a pivot axis PV of the lever 806 may include a biasing member 1720 (e.g., spring). The biasing member 1720 may be a torsion spring. Consequently, the securing mechanism 800 may be biased to the open position due to the biasing member 1720. In this regard, a user opening the lever 806 may maintain the securing mechanism 800 in the open position, thereby allowing easy removal of the canister assembly 700.

Returning to FIG. 16A, if lever 806 is opened, the manifold 804 will be retracted away from inlets 706 and 708 in the direction of arrow A, allowing canister assembly 700 to be pulled out of oxygen concentrator 100 in the direction of arrow B. Likewise, while lever 806 is in the open position, canister assembly 700 can be re-inserted (or a replacement canister assembly 700 can be inserted) into oxygen concentrator 100. By virtue of the central axes of outlets 710 and 712, as well as the central axes of the couplings 1608-1, 1608-2 of the manifold 1606, being generally parallel or substantially parallel to the direction in which the canister assembly 700 is inserted, pushing canister assembly 700 fully into oxygen concentrator 100 will bring outlets 710 and 712 into a pneumatic sealing engagement with manifold 1606. Once the outlets 710 and 712 are engaged with the manifold 1606, the canister assembly is constrained to be able to move only along the axis lines A-A within the compartment 1602. Similarly, by closing lever 806, the manifold 804 will slide into engagement with air inlets 706 and 708. By virtue of the central axes of inlets 706 and 708, as well as the central axes of the couplings 1708-1, 1708-2 of the manifold 804, being generally parallel or substantially parallel to the direction of movement of the manifold 804, movement of the manifold 804 brings the inlets into a pneumatic sealing engagement with manifold 804.

In addition, by virtue of the central axes of air inlets 706 and 708 being perpendicular or substantially perpendicular to the direction in which canister assembly 700 is inserted into the oxygen concentrator 100, the engagement (e.g., insertion engagement) between manifold 804 and inlets 706 and 708 may also serve to complete the securing of the canister assembly 700 in oxygen concentrator 100 when lever 806 is closed. In this regard, the structure of the movable manifold 804 may impede translation of the canister assembly along axis A-A as a result of the engagement of the movable manifold structure that moves to engage with the canister assembly along an axis different from the A-A axis (e.g., the B-B axis).

In some implementations, canisters 702 and 704 will be identical or substantially identical in size, and canister assembly 700 will be symmetrical about a plane which divides canisters 702 and 704. In some implementations, the installed position of canister assembly 700 may further be symmetrical about a plane which equally divides the inlet manifold, the outlet manifold and the canister assembly 700. Making canister assembly 700 symmetrical in this regard, and installing it symmetrically within oxygen concentrator 100 can help to balance the weight of the oxygen concentrator 100 such that there is symmetrical weight along the symmetry line SL, keeping the center of gravity at the center of the oxygen concentrator 100, which may contribute to its portability. Furthermore, there may be a symmetrical air path so that the PSA cycle may achieve the targeted oxygen concentration (i.e. the end state) in a shorter amount of time, and achieve improved oxygen concentration over the long term. (The targeted oxygen concentration may be at least 93%, or in the range 93% to 95%.) The symmetrical air path may lead to pressure balance (or equal pressure) on both the canisters during the equalization phase of the PSA cycle. Advantageously, there is no need for software intervention to achieve the end state in a shorter amount of time and improved oxygen concentration. For example, as illustrated in FIG. 20, the structure of the canisters of the canister assembly 700 may be formed to have a substantially symmetric structure about a symmetry line SL representing the aforementioned plane as illustrated in FIG. 20. Thus, each canister may be substantially symmetrical with respect to the other on opposing sides of the plane represented by the symmetry line SL. Similarly, the structure of the inlet manifold may be formed to have a substantially symmetric structure about the same symmetry line SL. As illustrated, the pneumatic path of canister 702 on one side of the symmetry line SL may be identical to the pneumatic path of canister 704, wherein the pneumatic path of canister 702 comprises the inlet 706 and the outlet 710, while the pneumatic path of the canister 704 comprises the inlet 708 and the outlet 712.

For example, the removable canister assembly 700 may have a first canister and a second canister, each configured as a sieve bed. At least one manifold (such as manifold 804, manifold 1606) may be coupled to the removable canister assembly 700 and may be configured to direct pneumatic flow with each of the first canister and the second canister. The manifold (such as manifold 804) may include a first portion including a first pneumatic path pneumatically coupled with the first canister and a second portion including a second pneumatic path pneumatically coupled with the second canister. The manifold (such as manifold 804) and the removable canister assembly 700 may be substantially symmetrical about a common symmetry plane wherein the common symmetry plane is between the first canister and the second canister, and wherein the common symmetry plane is also between the first portion and the second portion. Moreover, the manifold (such as manifold 804) may include a first valve and a second valve and the common symmetry plane may also be between the first valve and the second valve.

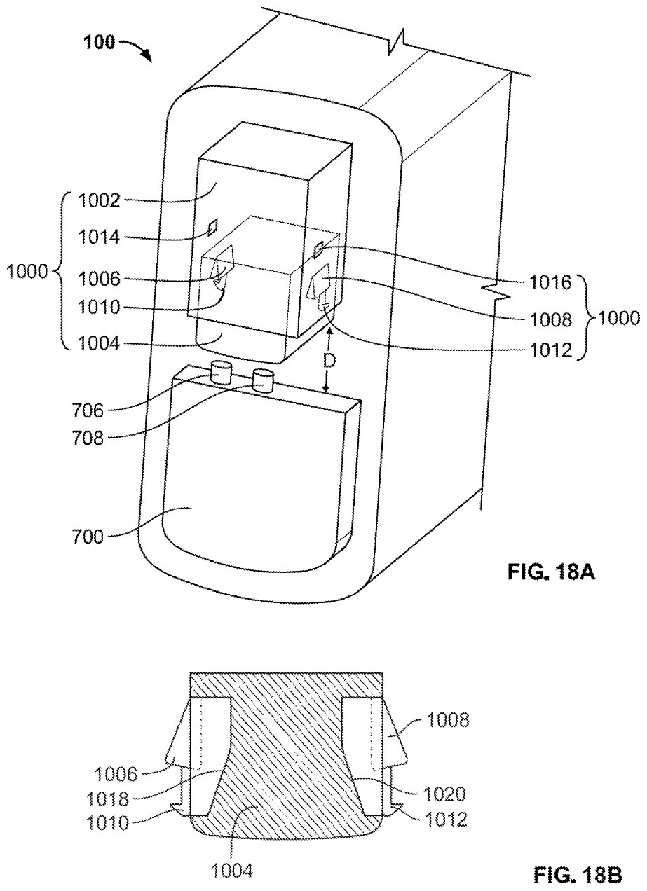

While FIGS. 16A, 16B, 17A-17D, illustrate example implementations of a securing mechanism 800, there are various alternative ways of creating the translational movement desired to extend and retract inlet manifold 804 so that it engages and secures canister assembly 700 within oxygen concentrator 100. For example, FIGS. 18A and 18B illustrate another implementation in which a securing mechanism 1000 includes a guide frame 1002, with two upper catches 1014 and 1016, and an inlet manifold 1004 with two latches 1006 and 1008 and two recesses 1018 and 1020. FIG. 18B is an isolated view of inlet manifold 1004, showing how latches 1006 and 1008 interface with recesses 1018 and 1020. As can be seen from FIGS. 18A and 18B, latches 1006 and 1008 each have tabs 1010 and 1012, respectively. By pressing the latches 1006 and 1008 into the recesses 1018 and 1020, respectively, manifold 1004 will be unlocked and free to translate within guide frame 1002. Manifold 1004 can thus be moved up or down by the user in the directions indicated by double-arrow D. When manifold 1004 is pushed down so that it fully engages air inlets 706 and 708, the tabs 1010 and 1012 of latches 1006 and 1008 will catch on the end of guide frame 1002 or a set of lower catches (not shown), locking manifold 1004 in place and securing canister assembly 700 in oxygen concentrator 100 as discussed above. Likewise, as manifold 1004 is translated away from canister assembly 700, the tabs 1010 and 1012 of latches 1006 and 1008 will eventually catch on upper catches 1014 and 1016, locking manifold 1004 in a position where canister assembly 700 can be removed from oxygen concentrator 100 as discussed above. Latches 1006 and 1008 may be biased or spring-loaded, or made of a resilient elastic material so that they maintain a bias to press outwardly toward side structure of the guide frame 1002.

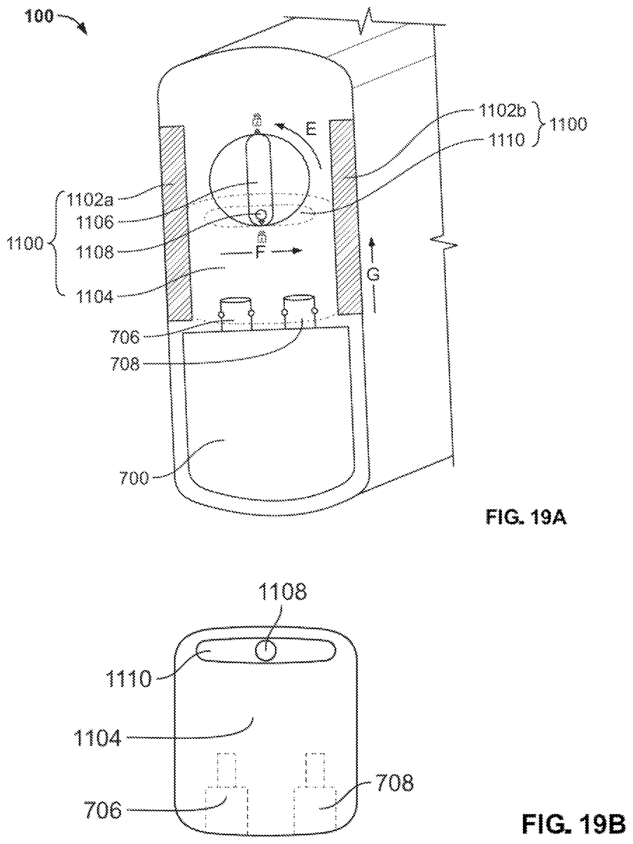

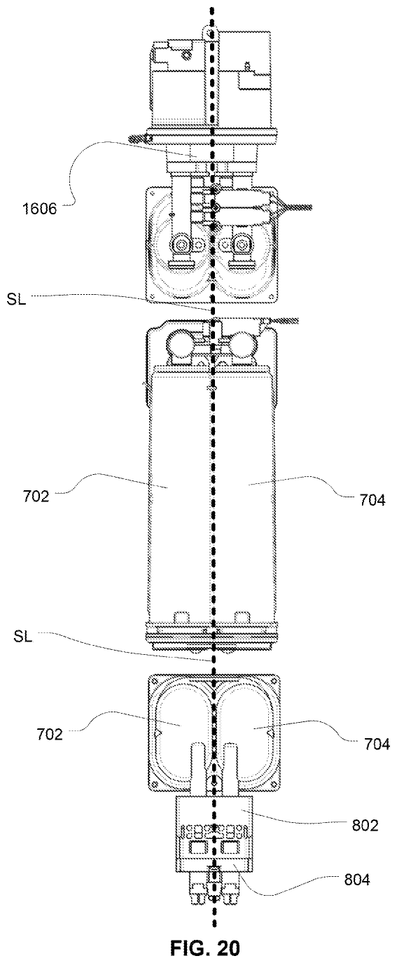

FIGS. 19A and 19B show another implementation in which securing mechanism 1100 includes linear guides 1102a and 1102b, a rotatable-type knob 1106 attached to a pin 1108, and an inlet manifold 1104 with a slot 1110. FIG. 19B is an isolated view of inlet manifold 1104, showing how slot 1110 interfaces with pin 1108. Rotation of the knob 1106 in one direction will retract the manifold 1104 away from the canister assembly 700 and rotation of the knob 1106 in the other direction will extend the manifold 1104 toward the canister assembly 700 as previously described in relation to the prior implementations. For example, the combination of knob 1106 and pin 1108 may form a cam, such that when knob 1106 is rotated in the direction of arrow E (e.g., anti-clockwise), pin 1108 will move within slot 1110 in the direction of arrow F, causing manifold 1104 to move along linear guides 1102a and 1102b, raising manifold 1104 away from canister assembly 700 in the direction of arrow G, such as until pin 1108 reaches the right-side of slot 1110 or the knob 1106 is rotated 180 degrees. Conversely, when pin 1108 is toward the right-side of slot 1110, rotating knob 1106 in the direction opposite arrow E (e.g., clockwise direction) will cause manifold 1104 to be pushed downwards and towards canister assembly 700 so that it engages air inlets 706 and 708. As will be understood, as FIGS. 19A and 19B show that slot 1110 extends to the right and left of pin 1108 (when manifold 1104 is engaged with canister assembly 700), knob 1106 may alternatively be rotated in the opposite direction from arrow E (e.g., clockwise direction) to raise manifold 1104 away from canister assembly 700, and in the direction of arrow E (once pin 1108 is at the left-most end of slot 1110) to push manifold 1104 back down toward canister assembly 700 so that it engages air inlets 706 and 708. In some implementations, slot 1110 may be half as wide as that shown in FIGS. 19A and 19B, such that knob 1106 can only be rotated in one direction in order to raise manifold 1104 away from canister assembly 700. In some implementations, slot 1110 may have one or more detents in order to aid in holding manifold 1104 in certain positions, e.g., in the fully raised or fully lowered positions.

In addition to the above, there are numerous ways in which an electromechanical mechanism may be used in place of a mechanical securing mechanism to move the inlet manifold 804 into and out of engagement with canister assembly 700.

### Outlet System

An outlet system, coupled to one or more of the canisters, includes one or more conduits for providing oxygen enriched air to a user. In an implementation, oxygen enriched air produced in either of canisters 302 and 304 is collected in accumulator 106 through check valves 142 and 144, respectively, as depicted schematically in FIG. 6. The oxygen enriched air leaving the canisters may be collected in the accumulator 106 prior to being provided to a user. In some implementations, a tube may be coupled to the accumulator 106 to provide the oxygen enriched air to the user. Oxygen enriched air may be provided to the user through an airway delivery device that transfers the oxygen enriched air to the user's mouth and/or nose. In an implementation, an outlet may include a tube that directs the oxygen toward a user's nose and/or mouth that may not be directly coupled to the user's nose.

Turning to FIG. 6, a schematic diagram of an implementation of an outlet system for an oxygen concentrator is shown. A supply valve 160 may be coupled to an outlet tube to control the release of the oxygen enriched air from accumulator 106 to the user. In an implementation, supply valve 160 is an electromagnetically actuated plunger valve. Supply valve 160 is actuated by controller 400 to control the delivery of oxygen enriched air to a user. Actuation of supply valve 160 is not timed or synchronized to the pressure swing adsorption process. Instead, actuation is synchronized to the user's breathing as described below. In some implementations, supply valve 160 may have continuously-valued actuation to establish a clinically effective amplitude profile for providing oxygen enriched air.

Oxygen enriched air in accumulator 106 passes through supply valve 160 into expansion chamber 162 as depicted in FIG. 6. In an implementation, expansion chamber 162 may include one or more devices configured to estimate an oxygen concentration of gas passing through the expansion chamber 162. Oxygen enriched air in expansion chamber 162 builds briefly, through release of gas from accumulator 106 by supply valve 160, and then is bled through a small orifice flow restrictor 175 to a flow rate sensor 185 and then to particulate filter 187. Flow restrictor 175 may be a 0.025 D flow restrictor. Other flow restrictor types and sizes may be used. In some implementations, the diameter of the air pathway in the housing may be restricted to create restricted gas flow. Optional flow rate sensor 185 may be any sensor configured to generate a signal representing the rate of gas flowing through the conduit. Particulate filter 187 may be used to filter bacteria, dust, granule particles, etc., prior to delivery of the oxygen enriched air to the user. The oxygen enriched air passes through filter 187 to connector 190 which sends the oxygen enriched air to the user via delivery conduit 192 and to pressure sensor 194.

The fluid dynamics of the outlet pathway, coupled with the programmed actuations of supply valve 160, may result in a bolus of oxygen being provided at the correct time and with an amplitude profile that assures rapid delivery into the user's lungs without excessive waste.

Expansion chamber 162 may include one or more oxygen sensors adapted to determine an oxygen concentration of gas passing through the chamber. In an implementation, the oxygen concentration of gas passing through expansion chamber 162 is estimated using an oxygen sensor 165. An oxygen sensor is a device configured to measure oxygen concentration in a gas. Examples of oxygen sensors include, but are not limited to, ultrasonic oxygen sensors, electrical oxygen sensors, chemical oxygen sensors, and optical oxygen sensors. In one implementation, oxygen sensor 165 is an ultrasonic oxygen sensor that includes an ultrasonic emitter 166 and an ultrasonic receiver 168. In some implementations, ultrasonic emitter 166 may include multiple ultrasonic emitters and ultrasonic receiver 168 may include multiple ultrasonic receivers. In implementations having multiple emitters/receivers, the multiple ultrasonic emitters and multiple ultrasonic receivers may be axially aligned (e.g., across the gas flow path which may be perpendicular to the axial alignment).

In use, an ultrasonic sound wave from emitter 166 may be directed through oxygen enriched air disposed in chamber 162 to receiver 168. The ultrasonic oxygen sensor 165 may be configured to detect the speed of sound through the oxygen enriched air to determine the composition of the oxygen enriched air. The speed of sound is different in nitrogen and oxygen, and in a mixture of the two gases, the speed of sound through the mixture may be an intermediate value proportional to the relative amounts of each gas in the mixture. In use, the sound at the receiver 168 is slightly out of phase with the sound sent from emitter 166. This phase shift is due to the relatively slow velocity of sound through a gas medium as compared with the relatively fast speed of the electronic pulse through wire. The phase shift, then, is proportional to the distance between the emitter and the receiver and inversely proportional to the speed of sound through the expansion chamber 162. The density of the gas in the chamber affects the speed of sound through the expansion chamber and the density is proportional to the ratio of oxygen to nitrogen in the expansion chamber. Therefore, the phase shift can be used to measure the concentration of oxygen in the expansion chamber. In this manner the relative concentration of oxygen in the accumulator may be estimated as a function of one or more properties of a detected sound wave traveling through the accumulator.

In some implementations, multiple emitters 166 and receivers 168 may be used. The readings from the emitters 166 and receivers 168 may be averaged to reduce errors that may be inherent in turbulent flow systems. In some implementations, the presence of other gases may also be detected by measuring the transit time and comparing the measured transit time to predetermined transit times for other gases and/or mixtures of gases.

The sensitivity of the ultrasonic sensor system may be increased by increasing the distance between the emitter 166 and receiver 168, for example to allow several sound wave cycles to occur between emitter 166 and the receiver 168. In some implementations, if at least two sound cycles are present, the influence of structural changes of the transducer may be reduced by measuring the phase shift relative to a fixed reference at two points in time. If the earlier phase shift is subtracted from the later phase shift, the shift caused by thermal expansion of expansion chamber 162 may be reduced or cancelled. The shift caused by a change of the distance between the emitter 166 and receiver 168 may be approximately the same at the measuring intervals, whereas a change owing to a change in oxygen concentration may be cumulative. In some implementations, the shift measured at a later time may be multiplied by the number of intervening cycles and compared to the shift between two adjacent cycles. Further details regarding sensing of oxygen in the expansion chamber may be found, for example, in U.S. Published Patent Application No. 2009-0065007, published Mar. 12, 2009, and entitled “Oxygen Concentrator Apparatus and Method”, which is incorporated herein by reference.

Flow rate sensor 185 may be used to determine the flow rate of gas flowing through the outlet system. Flow rate sensors that may be used include, but are not limited to: diaphragm/bellows flow meters; rotary flow meters (e.g. Hall effect flow meters); turbine flow meters; orifice flow meters; and ultrasonic flow meters. Flow rate sensor 185 may be coupled to controller 400. The rate of gas flowing through the outlet system may be an indication of the breathing volume of the user. Changes in the flow rate of gas flowing through the outlet system may also be used to determine a breathing rate of the user. Controller 400 may generate a control signal or trigger signal to control actuation of supply valve 160. Such control of actuation of the supply valve may be based on the breathing rate and/or breathing volume of the user, as estimated by flow rate sensor 185.

In some implementations, ultrasonic sensor 165 and, for example, flow rate sensor 185 may provide a measurement of an actual amount of oxygen being provided. For example, flow rate sensor 185 may measure a volume of gas (based on flow rate) provided and ultrasonic sensor 165 may provide the concentration of oxygen of the gas provided. These two measurements together may be used by controller 400 to determine an approximation of the actual amount of oxygen provided to the user.

Oxygen enriched air passes through flow rate sensor 185 to filter 187. Filter 187 removes bacteria, dust, granule particles, etc. prior to providing the oxygen enriched air to the user. The filtered oxygen enriched air passes through filter 187 to connector 190. Connector 190 may be a “Y” connector coupling the outlet of filter 187 to pressure sensor 194 and delivery conduit 192. Pressure sensor 194 may be used to monitor the pressure of the gas passing through conduit 192 to the user. In some implementations, pressure sensor 194 is configured to generate a signal that is proportional to the amount of positive or negative pressure applied to a sensing surface. Changes in pressure, sensed by pressure sensor 194, may be used to determine a breathing rate of a user, as well as the onset of inhalation (also referred to as the trigger instant) as described below. Controller 400 may control actuation of supply valve 160 based on the breathing rate and/or onset of inhalation of the user. In an implementation, controller 400 may control actuation of supply valve 160 based on information provided by either or both of the flow rate sensor 185 and the pressure sensor 194.

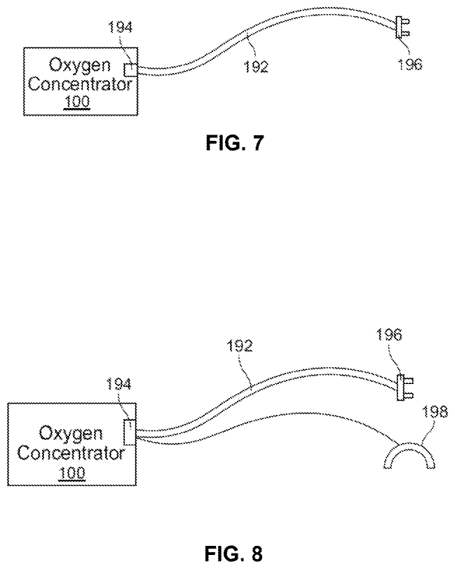

Oxygen enriched air may be provided to a user through conduit 192. In an implementation, conduit 192 may be a silicone tube. Conduit 192 may be coupled to a user using an airway delivery device 196, as depicted in FIGS. 7 and 8. Airway delivery device 196 may be any device capable of providing the oxygen enriched air to nasal cavities or oral cavities. Examples of airway delivery devices include, but are not limited to: nasal masks, nasal pillows, nasal prongs, nasal cannulas, and mouthpieces. A nasal cannula airway delivery device 196 is depicted in FIG. 7. Airway delivery device 196 is positioned proximate to a user's airway (e.g., proximate to the user's mouth and or nose) to allow delivery of the oxygen enriched air to the user while allowing the user to breathe air from the surroundings.

In an alternate implementation, a mouthpiece may be used to provide oxygen enriched air to the user. As shown in FIG. 8, a mouthpiece 198 may be coupled to oxygen concentrator 100. Mouthpiece 198 may be the only device used to provide oxygen enriched air to the user, or a mouthpiece may be used in combination with a nasal airway delivery device 196 (e.g., a nasal cannula). As depicted in FIG. 8, oxygen enriched air may be provided to a user through both a nasal airway delivery device 196 and a mouthpiece 198.

Mouthpiece 198 is removably positionable in a user's mouth. In one implementation, mouthpiece 198 is removably couplable to one or more teeth in a user's mouth. During use, oxygen enriched air is directed into the user's mouth via the mouthpiece. Mouthpiece 198 may be a night guard mouthpiece which is molded to conform to the user's teeth. Alternatively, mouthpiece may be a mandibular repositioning device. In an implementation, at least a majority of the mouthpiece is positioned in a user's mouth during use.

During use, oxygen enriched air may be directed to mouthpiece 198 when a change in pressure is detected proximate to the mouthpiece. In one implementation, mouthpiece 198 may be coupled to a pressure sensor 194. When a user inhales air through the user's mouth, pressure sensor 194 may detect a drop in pressure proximate to the mouthpiece. Controller 400 of oxygen concentrator 100 may control release of a bolus of oxygen enriched air to the user at the onset of inhalation.

During typical breathing of an individual, inhalation may occur through the nose, through the mouth or through both the nose and the mouth. Furthermore, breathing may change from one passageway to another depending on a variety of factors. For example, during more active activities, a user may switch from breathing through their nose to breathing through their mouth, or breathing through their mouth and nose. A system that relies on a single mode of delivery (either nasal or oral), may not function properly if breathing through the monitored pathway is stopped. For example, if a nasal cannula is used to provide oxygen enriched air to the user, an inhalation sensor (e.g., a pressure sensor or flow rate sensor) is coupled to the nasal cannula to determine the onset of inhalation. If the user stops breathing through their nose, and switches to breathing through their mouth, the oxygen concentrator 100 may not know when to provide the oxygen enriched air since there is no feedback from the nasal cannula. Under such circumstances, oxygen concentrator 100 may increase the flow rate and/or increase the frequency of providing oxygen enriched air until the inhalation sensor detects an inhalation by the user. If the user switches between breathing modes often, the default mode of providing oxygen enriched air may cause the oxygen concentrator 100 to work harder, limiting the portable usage time of the system.

In an implementation, a mouthpiece 198 is used in combination with a nasal airway delivery device 196 (e.g., a nasal cannula) to provide oxygen enriched air to a user, as depicted in FIG. 8. Both mouthpiece 198 and nasal airway delivery device 196 are coupled to an inhalation sensor. In one implementation, mouthpiece 198 and nasal airway delivery device 196 are coupled to the same inhalation sensor. In an alternate implementation, mouthpiece 198 and nasal airway delivery device 196 are coupled to different inhalation sensors. In either implementation, the inhalation sensor(s) may detect the onset of inhalation from either the mouth or the nose. Oxygen concentrator 100 may be configured to provide oxygen enriched air to the delivery device (i.e. mouthpiece 198 or nasal airway delivery device 196) proximate to which the onset of inhalation was detected. Alternatively, oxygen enriched air may be provided to both mouthpiece 198 and nasal airway delivery device 196 if onset of inhalation is detected proximate either delivery device. The use of a dual delivery system, such as depicted in FIG. 8 may be particularly useful for users when they are sleeping and may switch between nose breathing and mouth breathing without conscious effort.

### Controller System

Operation of oxygen concentrator 100 may be performed automatically using an internal controller 400 coupled to various components of the oxygen concentrator 100, as described herein. Controller 400 includes one or more processors 410 and internal memory 420, as depicted in FIG. 2. Methods used to operate and monitor oxygen concentrator 100 may be implemented by program instructions stored in internal memory 420 or an external memory medium coupled to controller 400, and executed by one or more processors 410. A memory medium may include any of various types of memory devices or storage devices. The term “memory medium” is intended to include an installation medium, e.g., a Compact Disc Read Only Memory (CD-ROM), floppy disks, or tape device; a computer system memory or random access memory such as Dynamic Random Access Memory (DRAM), Double Data Rate Random Access Memory (DDR RAM), Static Random Access Memory (SRAM), Extended Data Out Random Access Memory (EDO RAM), Random Access Memory (RAM), etc.; or a non-volatile memory such as a magnetic medium, e.g., a hard drive, or optical storage. The memory medium may comprise other types of memory as well, or combinations thereof. In addition, the memory medium may be located proximate to the controller 400 by which the programs are executed, or may be located in an external computing device that connects to the controller 400 over a network, such as the Internet. In the latter instance, the external computing device may provide program instructions to the controller 400 for execution. The term “memory medium” may include two or more memory media that may reside in different locations, e.g., in different computing devices that are connected over a network.

In some implementations, controller 400 includes processor 410 that includes, for example, one or more field programmable gate arrays (FPGAs), microcontrollers, etc. included on a circuit board disposed in oxygen concentrator 100. Processor 410 is configured to execute programming instructions stored in memory 420. In some implementations, programming instructions may be built into processor 410 such that a memory external to the processor 410 may not be separately accessed (i.e., the memory 420 may be internal to the processor 410).

Processor 410 may be coupled to various components of oxygen concentrator 100, including, but not limited to compression system 200, one or more of the valves used to control fluid flow through the system (e.g., valves 122, 124, 132, 134, 152, 154, 160), oxygen sensor 165, pressure sensor 194, flow rate sensor 185, temperature sensors (not shown), fan 172, and any other component that may be electrically controlled. In some implementations, a separate processor (and/or memory) may be coupled to one or more of the components.

Controller 400 is configured (e.g., programmed by program instructions) to operate oxygen concentrator 100 and is further configured to monitor the oxygen concentrator 100 such as for malfunction states or other process information. For example, in one implementation, controller 400 is programmed to trigger an alarm if the system is operating and no breathing is detected by the user for a predetermined amount of time. For example, if controller 400 does not detect a breath for a period of 75 seconds, an alarm LED may be lit and/or an audible alarm may be sounded. If the user has truly stopped breathing, for example, during a sleep apnea episode, the alarm may be sufficient to awaken the user, causing the user to resume breathing. The action of breathing may be sufficient for controller 400 to reset this alarm function. Alternatively, if the system is accidentally left on when delivery conduit 192 is removed from the user, the alarm may serve as a reminder for the user to turn oxygen concentrator 100 off.

Controller 400 is further coupled to oxygen sensor 165, and may be programmed for continuous or periodic monitoring of the oxygen concentration of the oxygen enriched air passing through expansion chamber 162. A minimum oxygen concentration threshold may be programmed into controller 400, such that the controller lights an LED visual alarm and/or an audible alarm to warn the user of the low concentration of oxygen.

Controller 400 is also coupled to internal power supply 180 and may be configured to monitor the level of charge of the internal power supply. A minimum voltage and/or current threshold may be programmed into controller 400, such that the controller lights an LED visual alarm and/or an audible alarm to warn the user of low power condition. The alarms may be activated intermittently and at an increasing frequency as the battery approaches zero usable charge.

The controller of the POC may implement compressor control to regulate pressure in the system. Thus, the POC may be equipped with a pressure sensor such as in the accumulator downstream of the sieve beds. The controller 400 in the POC 100 can control adjusting of the speed of the compressor using signals from the pressure sensor as well as a motor speed sensor such as in one or more modes. In this regard, the controller may implement dual control modes, designated a coarse pressure regulation mode and a fine pressure regulation mode. The coarse pressure regulation mode may be implemented for changing between the different flow rate settings (or “flow settings”) of the POC and for starting/initial activation. The fine pressure regulation mode may then take over upon completion of each operation of the coarse pressure regulation mode.

Additionally, the controller of the POC may be configured to implement bolus control to regulate bolus size in the system, which may optionally be implemented without use of a flow rate sensor of the POC. For example, the POC may be equipped with a pressure sensor, such as in the accumulator downstream of the sieve beds, and regulate bolus size, generated by the POC, as a function of pressure. Such regulation of bolus size may be a function of pressure and valve timing.

### Control Panel

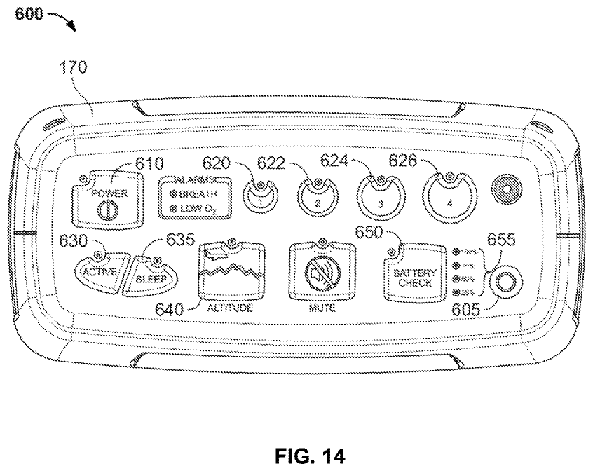

Control panel 600 serves as an interface between a user and controller 400 to allow the user to initiate predetermined operation modes of the oxygen concentrator 100 and to monitor the status of the system. FIG. 14 depicts an implementation of control panel 600. Charging input port 605, for charging the internal power supply 180, may be disposed in control panel 600.

In some implementations, control panel 600 may include buttons to activate various operation modes for the oxygen concentrator 100. For example, control panel may include power button 610, flow rate setting buttons 620 to 626, active mode button 630, sleep mode button 635, altitude button 640, and a battery check button 650. In some implementations, one or more of the buttons may have a respective LED that may illuminate when the respective button is pressed, and may power off when the respective button is pressed again. Power button 610 may power the system on or off. If the power button is activated to turn the system off, controller 400 may initiate a shutdown sequence to place the system in a shutdown state (e.g., a state in which both canisters are pressurized). Flow rate setting buttons 620, 622, 624, and 626 allow a flow rate of oxygen enriched air to be selected (e.g., 0.2 LPM by button 620, 0.4 LPM by button 622, 0.6 LPM by button 624, and 0.8 LPM by button 626). Altitude button 640 may be activated when a user is going to be in a location at a higher elevation than the oxygen concentrator 100 is regularly used by the user.

Battery check button 650 initiates a battery check routine in the oxygen concentrator 100 which results in a relative battery power remaining LED 655 being illuminated on control panel 600.

A user may have a low breathing rate or depth if relatively inactive (e.g., asleep, sitting, etc.) as estimated by comparing the detected breathing rate or depth to a threshold. The user may have a high breathing rate or depth if relatively active (e.g., walking, exercising, etc.). An active/sleep mode may be estimated automatically and/or the user may manually indicate active mode or sleep mode by pressing button 630 for active mode or button 635 for sleep mode.

### Methods of Operating the POC

The methods of operating and monitoring the POC 100 described below may be executed by the one or more processors, such as the one or more processors 410 of the controller 400, configured by program instructions, such as including, as previously described, the one or more functions and/or associated data corresponding thereto, stored in a memory such as the memory 420 of the POC 100. Alternatively, some or all of the steps of the described methods may be similarly executed by one or more processors of an external computing device to which the controller is connected via the transceiver 430. In this latter implementation, the processors 410 may be configured by program instructions stored in the memory 420 of the POC 100 to transmit to the external computing device the measurements and parameters necessary for the performance of those steps that are to be carried out at the external computing device.

The main use of an oxygen concentrator 100 is to provide supplemental oxygen to a user. One or more flow rate settings may be selected on a control panel 600 of the oxygen concentrator 100, which then will control operations to achieve production of the oxygen enriched air according to the selected flow rate setting. In some implementations, a plurality of flow rate settings may be implemented (e.g., five flow rate settings). As described in more detail herein, the controller may implement a POD (pulsed oxygen delivery) or demand mode of operation to regulate size of one or more released boluses to achieve delivery of the oxygen enriched air according to the selected flow rate setting.

In order to maximise the effect of the delivered oxygen enriched air, controller 400 may be programmed to synchronise release of each bolus of the oxygen enriched air with the user's inhalations. Releasing a bolus of oxygen enriched air to the user as the user inhales may prevent wastage of oxygen by not releasing oxygen, for example, when the user is exhaling. For concentrators that operate in POD mode, the flow rate settings on the control panel 600 may correspond to minute volumes (bolus volume multiplied by breathing rate per minute) of delivered oxygen, e.g. 0.2 LPM, 0.4 LPM, 0.6 LPM, 0.8 LPM, 1.1 LPM.

Oxygen enriched air produced by oxygen concentrator 100 is stored in an oxygen accumulator 106 and, in POD mode, released to the user as the user inhales. The amount of oxygen enriched air provided by the oxygen concentrator 100 is controlled, in part, by supply valve 160. In an implementation, supply valve 160 is opened for a sufficient amount of time to provide the appropriate amount of oxygen enriched air, as estimated by controller 400, to the user. In order to minimize the wastage of oxygen, the oxygen enriched air may be provided as a bolus soon after the onset of a user's inhalation is detected. For example, the bolus of oxygen enriched air may be provided in the first few milliseconds of a user's inhalation.

In an implementation, pressure sensor 194 may be used to determine the onset of inhalation by the user. For example, the user's inhalation may be detected by using pressure sensor 194. In use, conduit 192 for providing oxygen enriched air is coupled to a user's nose and/or mouth through the nasal airway delivery device 196 and/or mouthpiece 198. The pressure in conduit 192 is therefore representative of the user's airway pressure and therefore indicative of user respiration. At the onset of an inhalation, the user begins to draw air into their body through the nose and/or mouth. As the air is drawn in, a negative pressure is generated at the end of the conduit 192, due, in part, to the venturi action of the air being drawn across the end of the conduit. Controller 400 analyses the pressure signal from the pressure sensor 194 to detect a drop in pressure indicating the onset of inhalation. Upon detection of the onset of inhalation, supply valve 160 is opened to release a bolus of oxygen enriched air from the accumulator 106. A positive change or rise in the pressure indicates an exhalation by the user, upon which the release of oxygen enriched air is discontinued. In one implementation, when a positive pressure change is sensed, supply valve 160 is closed until the next onset of inhalation is detected. Alternatively, supply valve 160 may be closed after a predetermined interval known as the bolus duration. By measuring the intervals between adjacent onsets of inhalation, the user's breathing rate may be estimated. By measuring the intervals between onsets of inhalation and the subsequent onsets of exhalation, the user's inspiratory time may be estimated. Thus, the user's breathing rate or respiration rate may be detected with a signal from the pressure sensor and/or a flow rate sensor.

In other implementations, the pressure sensor 194 may be located in a sensing conduit that is in pneumatic communication with the user's airway, but separate from the delivery conduit 192. In such implementations the pressure signal from the pressure sensor 194 is therefore also representative of the user's airway pressure.

In some implementations, the sensitivity of the pressure sensor 194 may be affected by the physical distance of the pressure sensor 194 from the user, especially if the pressure sensor 194 is located in oxygen concentrator 100 and the pressure difference is detected through the conduit 192 coupling the oxygen concentrator 100 to the user. In some implementations, the pressure sensor 194 may be placed in the airway delivery device 196 used to provide the oxygen enriched air to the user. A signal from the pressure sensor 194 may be provided to controller 400 in the oxygen concentrator 100 electronically via a wire or through telemetry such as through Bluetooth™ or other wireless technology.

In some implementations, if the user's current activity level, such as that estimated using the detected user's breathing rate, exceeds a predetermined threshold, controller 400 may implement an alarm (e.g., visual and/or audio) to warn the user that the current breathing rate is exceeding the delivery capacity of the oxygen concentrator 100. For example, the threshold may be set at 40 breaths per minute (BPM).

### Glossary

For the purposes of the present technology disclosure, in certain forms of the present technology, one or more of the following definitions may apply. In other forms of the present technology, alternative definitions may apply.

Air: In certain forms of the present technology, air may be taken to mean atmospheric air, consisting of 78% nitrogen (N₂), 21% oxygen (O₂), and 1% water vapour, carbon dioxide (CO₂), argon (Ar), and other trace gases.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Ambient: In certain forms of the present technology, the term ambient will be taken to mean (i) external of the treatment system or user, and (ii) immediately surrounding the treatment system or user.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

Patient: A person, whether or not they are suffering from a respiratory disorder.

Pressure: Force per unit area. Pressure may be expressed in a range of units, including cmH₂O, g-f/cm2 and hectopascal. 1 cmH₂O is equal to 1 g-f/cm2 and is approximately 0.98 hectopascal (1 hectopascal=100 Pa=100 N/m²=1 millibar˜0.001 atm). In this specification, unless otherwise stated, pressure is given in units of cmH₂O.

Port, orifice opening: “port”, “orifice” and “opening” are used interchangeably.

### General Remarks

The term “coupled” as used herein means either a direct connection or an indirect connection (e.g., one or more intervening connections) between one or more objects or components. The phrase “connected” means a direct connection between objects or components such that the objects or components are connected directly to each other. As used herein the phrase “obtaining” a device means that the device is either purchased or constructed.

In the present disclosure, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

### Other Remarks

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately. It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology. Further modifications and alternative implementations of various aspects of the present technology may be apparent to those skilled in the art in view of this description. Accordingly, this description is to be construed as illustrative only and is for the purpose of teaching those skilled in the art the general manner of carrying out the technology. It is to be understood that the forms of the technology shown and described herein are to be taken as implementations. Elements and materials may be substituted for those illustrated and described herein, parts and processes may be reversed, and certain features of the technology may be utilized independently, all as would be apparent to one skilled in the art after having the benefit of this description of the technology. Changes may be made in the elements described herein without departing from the spirit and scope of the technology as described in the appended claims.

Label List

| | |
|---|---|
| oxygen concentrator | 100 |
| inlet | 101 |
| compression system inlets | 105 |
| inlet | 105 |
| accumulator | 106 |
| accumulator pressure sensor | 107 |
| muffler | 108 |
| valves | 122 |
| valves | 124 |
| filter | 129 |
| outlet | 130 |
| valves | 132 |
| muffler | 133 |
| valves | 134 |
| hundred thirty-five | 135 |
| spring baffle | 139 |
| baffle | 139 |
| check valve | 142 |
| check valve | 144 |
| flow restrictor | 151 |
| valve | 152 |
| flow restrictor | 153 |
| valve | 154 |
| flow restrictor | 155 |
| supply valve | 160 |
| expansion chamber | 162 |
| oxygen sensor | 165 |
| emitter | 166 |
| receiver | 168 |
| outer housing | 170 |
| fan | 172 |
| outlet | 173 |
| outlet port | 174 |
| flow restrictor | 175 |
| power supply | 180 |
| flow rate sensor | 185 |
| filter | 187 |
| connector | 190 |
| conduit | 192 |
| pressure sensor | 194 |
| airway delivery device | 196 |
| mouthpiece | 198 |
| compression system | 200 |
| speed sensor | 201 |
| compressor | 210 |
| compressor outlet | 212 |
| motor | 220 |
| armature | 230 |
| external armature | 230 |
| air transfer device | 240 |
| compressor outlet conduit | 250 |
| canister assembly | 300 |
| canister | 302 |
| canister | 304 |
| air inlet | 306 |
| housing | 310 |
| base | 315 |
| valve seats | 322 |
| outlet | 325 |
| gases | 327 |
| conduit | 330 |
| valve seats | 332 |
| apertures | 337 |
| conduit | 342 |
| conduit | 344 |
| conduit | 346 |
| opening | 375 |
| controller | 400 |
| processor | 410 |
| memory | 420 |
| transceiver | 430 |
| housing component | 510 |
| conduit | 530 |
| conduit | 532 |
| conduit | 534 |
| openings | 542 |
| opening | 544 |
| valve seat | 552 |
| valve seat | 554 |
| control panel | 600 |
| input port | 605 |
| power button | 610 |
| flow rate setting buttons | 620 |
| flow rate setting buttons | 622 |
| button | 624 |
| button | 626 |
| active mode button | 630 |
| mode button | 635 |
| altitude button | 640 |
| battery check button | 650 |
| LED | 655 |
| canister | 702 |
| canister | 704 |
| inlet | 706 |
| inlet | 708 |
| outlet | 710 |
| outlet | 712 |
| mechanism | 800 |
| guide frame | 802 |
| manifold | 804 |
| lever | 806 |
| linkage arm | 808 |
| slots | 810 |
| mechanism | 1000 |
| guide frame | 1002 |
| manifold | 1004 |
| latches | 1006 |
| tabs | 1010 |
| upper catches | 1014 |
| recesses | 1018 |
| mechanism | 1100 |
| manifold | 1104 |
| knob | 1106 |
| pin | 1108 |
| slot | 1110 |
| container portion | 1504 |
| cap portion | 1508 |
| flanges | 1510 |
| flange portion | 1511 |
| compartment | 1602 |
| portal | 1604 |
| manifold | 1606 |
| battery compartment | 1665 |
| lid | 1666 |
| lid | 1667 |
| indicator | 1669 |
| port | 1673 |
| removable panel | 1675 |
| cooling system inlets | 1677 |
| barb | 1714 |
| member | 1720 |
| valve mounting brackets | 1722 |
| channel | 706C |
| port | 706P |
| seal | 706S |
| channel | 708C |
| port | 708P |
| seal | 708S |
| channel | 710C |
| port | 710P |
| port | 712P |
| discrete recess | 1512A |
| discrete recess | 1512B |
| inlet groove | 1514A |
| inlet groove | 1514B |
| container volumes | 1506-1 |
| container volumes | 1506-2 |
| coupling | 1608-1 |
| coupling | 1608-2 |
| cooling system outlet | 1671-1 |
| cooling system outlet | 1671-2 |
| couplings | 1708-1 |
| outlet couplings | 1708-2 |
| first port | 1710-1 |
| second port | 1710-2 |
| grip handle | 1712-1 |
| grip handle | 1712-2 |
| valve | 1730-1 |
| valve | 1730-2 |

1. An oxygen concentrator comprising:
a first manifold;
a second manifold;
a compression system, including a motor operated compressor, configured to feed a feed gas for one or more sieve beds via the first manifold;
an accumulator configured to receive oxygen enriched air from the one or more sieve beds via the second manifold; and
an outer housing for the first manifold, the second manifold, the compression system, and the accumulator, the outer housing further comprising an external access portal to a compartment in the outer housing, the compartment for removably receiving the one or more sieve beds that are configured as a removable canister assembly;
wherein the first manifold is in the outer housing adjacent to the compartment, the first manifold having one or more inlet couplings for removably coupling respectively with one or more inlets of the removable canister assembly, the one or more inlet couplings each having a first central axis;
wherein the second manifold is in the outer housing adjacent to the compartment, the second manifold having one or more outlet couplings for removably coupling respectively with one or more outlets of the removable canister assembly, the one or more outlet couplings each having a second central axis; and
wherein the first central axis and the second central axis form an angle comprising any one of an obtuse angle, an acute angle, or a right angle.

2. The oxygen concentrator of claim 1 wherein the angle is in a range of approximately forty degrees to approximately one hundred and thirty-five degrees.

3. The oxygen concentrator of claim 2 wherein the angle is in a range of approximately fifty degrees to approximately one hundred and twenty-five degrees.

4. The oxygen concentrator of claim 3 wherein the angle is in a range of approximately sixty degrees to approximately one hundred and fifteen degrees.

5. The oxygen concentrator of claim 4 wherein the angle is in a range of approximately seventy degrees to approximately one hundred and five degrees.

6. The oxygen concentrator of claim 5 wherein the angle is approximately ninety degrees.

7. The oxygen concentrator of any one of claims 1 to 6 further comprising the removable canister assembly, the removable canister assembly comprising at least one canister, each canister containing gas separation adsorbent of one of the one or more sieve beds, each canister including:
an inlet in fluid communication with the contained gas separation adsorbent of the one of the one or more sieve beds;
an outlet in fluid communication with the contained gas separation adsorbent of the one of the one or more sieve beds; and
wherein the inlet and outlet are arranged at opposite ends of the canister, and wherein the inlet has a third central axis and the outlet has a fourth central axis, wherein the first central axis is aligned with the third central axis and the second central axis is aligned with the fourth central axis.

8. The oxygen concentrator of claim 7 wherein the removable canister assembly comprises a cap portion, the cap portion comprising a planar surface for covering one end of the at least one canister, the cap portion further comprising the inlet of the at least one canister, and wherein the third central axis of the inlet of each canister is generally parallel to the planar surface.

9. The oxygen concentrator of any one of claims 1 to 8, wherein the removable canister assembly comprises at least two container portions, each container portion defining a volume for containing gas separation adsorbent of the one or more sieve beds, wherein the container portions are separable.

10. The oxygen concentrator of any one of claims 1 to 8, wherein the removable canister assembly comprises at least two container portions, each container portion defining a volume for containing gas separation adsorbent of the one or more sieve beds, wherein the container portions are formed as a unitary structure.

11. The oxygen concentrator of any one of claims 1 to 10 wherein the one or more inlet couplings comprise a pneumatically sealable structure to complement a reciprocal structure of the inlet.

12. The oxygen concentrator of any one of claims 1 to 11 wherein the one or more outlet couplings comprise a pneumatically sealable structure to complement a reciprocal structure of the outlet.

13. The oxygen concentrator of any one of claims 1 to 12 wherein the first manifold is configured to move within the oxygen concentrator to couple and decouple with the inlet for insertion and removal of the removable canister assembly.

14. The oxygen concentrator of claim 13 wherein the first manifold includes one or more valves configured to move with the first manifold, each of the one or more valves being configured to gate fluid flow of one of the one or more inlet couplings.

15. The oxygen concentrator of any one of claims 13 to 14, further comprising:
a securing mechanism configured to reversibly engage the first manifold with the removable canister assembly for:
establishing pneumatic sealing between the first manifold and the removable canister assembly, and
securing the removable canister assembly within the oxygen concentrator.

16. The oxygen concentrator of claim 15 wherein the securing mechanism comprises linkage arms and a lever, wherein the linkage arms and the lever are configured to move the manifold within a guide frame of the oxygen concentrator.

17. The oxygen concentrator of any one of claims 15 to 16, wherein the securing mechanism further comprises one or more grip handles for user manipulation of the securing mechanism.

18. The oxygen concentrator of any one of claims 15 to 17, wherein the securing mechanism further comprises one or more latches for securing the manifold in pneumatic sealing with the removable canister assembly.

19. The oxygen concentrator of claim 15, wherein the securing mechanism comprises a knob for securing the manifold in pneumatic sealing with the removable canister assembly.

20. An oxygen concentrator comprising:
one or more sieve beds, each containing a gas separation adsorbent;
a compression system, including a motor operated compressor, configured to feed a feed gas into the one or more sieve beds;
an accumulator configured to receive oxygen enriched air from the one or more sieve beds;
wherein the one or more sieve beds are configured as a removable canister assembly comprising at least one canister, each canister containing the gas separation adsorbent of one of the one or more sieve beds, each canister of the removable canister assembly including:
an inlet in fluid communication with the contained gas separation adsorbent of the one of the one or more sieve beds; and
an outlet in fluid communication with the contained gas separation adsorbent of the one of the one or more sieve beds;
a manifold configured to pneumatically engage at least one inlet or outlet so as to enable fluid flow between the manifold and the at least one inlet or outlet; and
a securing mechanism configured to move the manifold into and out of pneumatic engagement with the at least one inlet or outlet, wherein moving the manifold into pneumatic engagement with the at least one inlet or outlet secures each canister within the oxygen concentrator.

21. The oxygen concentrator of claim 20 further comprising a guide frame for guiding movement of the manifold.

22. The oxygen concentrator of claim 21 wherein the securing mechanism comprises linkage arms and a lever configured to move the manifold within the guide frame of the oxygen concentrator.

23. The oxygen concentrator of any one of claims 20 to 22, wherein the securing mechanism further comprises one or more grip handles for user manipulation of the manifold.

24. The oxygen concentrator of any one of claims 20 to 21, wherein the securing mechanism further comprises one or more latches for securing the manifold in pneumatic engagement with the removable canister assembly.

25. The oxygen concentrator of any one of claims 20 to 21, wherein the securing mechanism further comprises a knob for securing the manifold in pneumatic engagement with the removable canister assembly.

26. The oxygen concentrator of any one of claims 20 to 25, wherein the manifold comprises a coupling having an orifice configured to receive, within the orifice, a nipple of the at least one inlet or outlet.

27. The oxygen concentrator of any one of claims 20 to 26 wherein the manifold includes one or more valves configured to move with the manifold, each of the one or more valves being configured to gate a pneumatic path of the manifold.

28. A removable canister assembly for an oxygen concentrator comprising:
one or more sieve beds configured as one or more canisters, each of the one or more canisters containing gas separation adsorbent, each canister including:
an inlet in fluid communication with the contained gas separation adsorbent; and
an outlet in fluid communication with the contained gas separation adsorbent;
wherein the inlet and outlet are arranged at opposite ends of the canister, and wherein the inlet has a first central axis and the outlet has a second central axis, and wherein the first central axis and the second central axis form one of an obtuse angle, an acute angle, or a right angle.

29. The removable canister assembly of claim 28 wherein the angle is in a range of approximately forty degrees to approximately one hundred and thirty-five degrees.

30. The removable canister assembly of claim 29 wherein the angle is in a range of approximately fifty degrees to approximately one hundred and twenty-five degrees.

31. The removable canister assembly of claim 30 wherein the angle is in a range of approximately sixty degrees to approximately one hundred and fifteen degrees.

32. The removable canister assembly of claim 31 wherein the angle is in a range of approximately seventy degrees to approximately one hundred and five degrees.

33. The removable canister assembly of claim 32 wherein the angle is approximately ninety degrees.

34. The removable canister assembly of any one of claims 28 to 33 wherein the inlet comprises a nipple with an inlet port.

35. The removable canister assembly of any one of claims 28 to 34 wherein the outlet comprises a nipple with an outlet port.

36. The removable canister assembly of any one of claims 28 to 35 further comprising a cap portion, the cap portion comprising a planar surface for covering one end of each of the one or more canisters, the cap portion further comprising the outlet of each canister, and wherein the second central axis of the outlet is generally parallel to the planar surface.

37. The removable canister assembly of any one of claims 28 to 35 further comprising a cap portion, the cap portion comprising a planar surface for covering one end of each of the one or more canisters, the cap portion further comprising the inlet of each canister, and wherein the first central axis of the inlet is generally parallel to the planar surface.

38. The canister assembly of any one of claims 28 to 37, further comprising at least two container portions, each container portion defining a volume for containing gas separation adsorbent, wherein the container portions are separable.

39. The canister assembly of any one of claims 28 to 37, further comprising at least two container portions, each container portion defining a volume for containing gas separation adsorbent, wherein the container portions are formed as a unitary structure.

40. A portable oxygen concentrator apparatus comprising:
user removable means for containing a gas separation adsorbent;
means for feeding a feed gas into the user removable means;
accumulation means for receiving oxygen enriched air from the user removable means; and
securing means for:
moving a manifold into and out of pneumatic engagement with at least one inlet or outlet of the user removable means; and
securing the user removable means within the portable oxygen concentrator apparatus for use.

41. A portable oxygen concentrator comprising:
a removable canister assembly comprising a first sieve bed and a second sieve bed, each sieve bed comprising a canister; and
a manifold configured to direct pneumatic flow with each of the first sieve bed and the sieve bed, the manifold comprising a first portion including a first pneumatic path pneumatically coupled with the first sieve bed and a second portion including a second pneumatic path pneumatically coupled with the second sieve bed,
wherein the manifold and the removable canister assembly are substantially symmetrical about a common symmetry plane, the common symmetry plane being (a) between the first sieve bed and the second sieve bed, and (b) between the first portion and the second portion of the manifold.

42. The portable oxygen concentrator of claim 41 wherein the manifold comprises a first valve and a second valve and wherein the common symmetry plane is also between the first valve and the second valve.
